(19) United States
(12) Patent Application Publication
(10) Pub. No.: US 2014/0286916 A1
(43) Pub. Date: Sep. 25, 2014

(54) Uses of Growth and Differentiation Factor 8 (GDF-8)

(71) Applicant: Bone Therapeutics S.A., Gosselies (BE)

(72) Inventors: Valentina Albarani, Bruxelles (BE); Enrico Bastianelli, Rhode-St-Genese (BE); Christelle Bizimungu, Bornival (BE); Alice Joly, Baisy Thy (BE); Xuan Mai Nguyen, Bruxelles (BE); Isabelle Tytgat, Bruxelles (BE)

(73) Assignee: Bone Therapeutics, Gosselies (BE)

(21) Appl. No.: 14/351,249

(22) PCT Filed: Oct. 10, 2012
(86) PCT No.: PCT/EP2012/069986

§ 371 Date: Apr. 11, 2014

(30) Foreign Application Priority Data
Oct. 11, 2011 (EP) 11184625.9

(51) Int. Cl. A61K 38/18; A61K 35/28

(52) U.S. Cl. CPC A61K 38/18; A61K 35/28; A61K 2035/122
USPC 424/93.7; 435/375; 435/366; 514/7.6

(57) ABSTRACT

The present invention relates to uses of growth and differentiation factor 8 (GDF-8) in vitro or in vivo for reducing the immunogenicity or risk of rejection of cells such as in particular mesenchymal stem cells (MSC), tissues or materials. The present invention further relates to methods for differentiating MSC in vitro or ex vivo into osteoprogenitors or osteoblastic cells or a cell population comprising osteoprogenitors and/or osteoblastic cells using FGF-2 and GDF-8. In addition, the present invention relates to osteoprogenitors or osteoblastic cells or a cell population comprising osteoprogenitors and/or osteoblastic cells obtainable by such methods and to the osteoprogenitors or osteoblastic cells or a cell population comprising osteoprogenitors and/or osteoblastic cells for use in the treatment of musculoskeletal diseases.

### FIELD

The present invention relates to applications of growth and differentiation factor 8 (GDF-8) in vitro or in vivo for altering properties of cells such as in particular mesenchymal stem cells (MSC), tissues or materials. The present invention further relates to methods for differentiating MSC in vitro or ex vivo into osteoprogenitors (comprising early and late osteoprogenitors) or osteoblastic cells (comprising pre-osteoblasts, osteoblasts and osteocytes) or a cell population comprising osteoprogenitors and/or osteoblastic cells. In addition, the present invention relates to osteoprogenitors or osteoblastic cells or a cell population comprising such osteoprogenitors and/or osteoblastic cells, obtainable by the methods, and to said osteoprogenitors or osteoblastic cells (such as for instance allogeneic osteoprogenitors or allogeneic osteoblasts) or the cell population comprising osteoprogenitors and/or osteoblastic cells for use in the treatment of musculoskeletal diseases.

### BACKGROUND

Musculoskeletal diseases or disorders can affect the bones, muscles, joints, cartilage, tendons, ligaments, and other connective tissue that supports and binds tissues and organs together. These diseases can develop over time or can result for instance by excessive use of the musculoskeletal system or from trauma. Musculoskeletal diseases can be difficult to diagnose and/or treat due to the close relation of the musculoskeletal system to other internal systems.

A possible and promising approach for the treatment of musculoskeletal diseases and in particular of bone diseases is transplantation of mesenchymal stem cells (MSC) capable of undergoing osteogenic differentiation or of cells that are committed towards osteoblastic lineage.

MSC have been used previously to treat bone disorders (Gangji et al. Expert Opin Biol Ther, 2005, vol. 5, 437-42). However, although such relatively undifferentiated stem cells can be transplanted, they are not committed to the osteoblastic lineage and therefore a considerable proportion of so-transplanted stem cells may not eventually contribute to the formation of the desired tissue. Moreover, the quantity of such stem cells obtainable from any possible source tissues is frequently unsatisfactory.

WO 2007/093431 disclosed a method aiming to achieve an adequate extent of in vitro expansion of isolated MSC and to yield cells that display osteoblastic phenotype. In said method, human MSC were particularly cultured in the presence of serum or plasma and basic fibroblast growth factor (FGF-2).

WO 2009/087213 described a method for osteogenic differentiation of human MSC, in particular using human plasma or serum, FGF-2 and TGFβ-1.

However, there exists a need for further and/or improved reliable methods for producing useful osteoprogenitors, or osteoblastic cells, or cell populations comprising osteoprogenitors and/or osteoblastic cells, from MSC.

There further exists a need for modifying cells, tissues or materials useful for administration to patients, such as in particular to reduce the immunogenicity or the risk of rejection thereof by the patients.

### SUMMARY

As exemplified in the experimental section, the inventors realised that growth and differentiation factor 8 (GDF-8) advantageously reduces immunogenicity of cells, such as for example mesenchymal stem cells (MSC), cultured or differentiated (e.g., differentiated into cells of mesenchymal cell lineages, such as among others into osteoprogenitors or osteoblastic cells or cell populations comprising osteoprogenitors and/or osteoblastic cells) in presence of GDF-8.

Expanding on the above-mentioned findings, the inventors recognized the ability of GDF-8 to reduce the immunogenicity of cells. Accordingly, an aspect of the invention relates to the use of growth and differentiation factor 8 (GDF-8) for reducing the immunogenicity of cells in vitro. Such use is advantageous because it allows transplantation of the cells for instance to an allogeneic subject. Particular embodiments provide the use of GDF-8 for reducing the immunogenicity of cells in vitro, wherein the cells are selected from the group consisting of mesenchymal stem cells (MSC), cells obtained by differentiation of MSC, cells of osteocytic lineage, cells of chondrocytic lineage, cells of adipocytic lineage, cells of myocytic lineage, cells of tendonocytic lineage, cells of fibroblastic lineage, and cells of stromogenic lineage. Cells of osteocytic, chondrocytic, adipocytic, myocytic, tendonocytic, fibroblastic, or stromogenic lineages may be considered to belong to the category of mesenchymal cell lineages, whereby the usefulness of GDF-8 for reducing immunogenicity of such cells is underscored.

In certain embodiments, the cells of osteocytic lineage, cells of chondrocytic lineage, cells of adipocytic lineage, cells of myocytic lineage, cells of tendonocytic lineage, cells of fibroblastic lineage, or cells of stromogenic lineage may be obtained by differentiation of MSC, more particularly the cells may be obtained by in vitro or ex vivo differentiation of MSC. Differentiation of MSC may involve culturing MSC under conditions capable of inducing the differentiation of MSC towards the desired cell type, more typically culturing MSC in a medium comprising one or more factors (e.g., growth factors) capable of inducing the differentiation of MSC towards the desired cell type. Protocols for differentiation of MSC are known per se (see, inter alia, WO 2007/093431; and further REGER, R. L. et al. ‘Differentiation and Characterization of Human MSCs’. In: Mesenchymal Stem Cells: Methods and Protocols (Methods in Molecular Biology), Edited by D. J. Prockop et al. Humana Press, 2008, Vol. 449, p. 93-107; VERMURI, M. C. et al. (Eds.). Mesenchymal Stem Cell Assays and Applications (Methods in Molecular Biology). Humana Press, 2011, Vol. 698, especially pages 201 to 352).

To reduce the immunogenicity of the cells obtained by in vitro or ex vivo differentiation of MSC, the cells may be exposed to (i.e., contacted with or cultured in a medium containing) GDF-8 after the desired cells have been obtained by differentiation of MSC, and/or the (MSC) cells may be exposed to GDF-8 as a part of one or more steps of the respective process or protocol to differentiate MSC into the desired cells. By means of an example, as discussed elsewhere in this specification, MSC may be differentiated into osteoprogenitors or osteoblastic cells or a cell population comprising osteoprogenitors and/or osteoblastic cells by a method comprising the step of culturing the MSC in a medium comprising plasma or serum, GDF-8 and fibroblast growth factor 2 (FGF-2), whereby the immunogenicity of the resultant cells is reduced.

Conversely, cells obtained by differentiation of MSC may thus particularly refer to cells of osteocytic lineage, cells of chondrocytic lineage, cells of adipocytic lineage, cells of myocytic lineage, cells of tendonocytic lineage, cells of fibroblastic lineage, or cells of stromogenic lineage.

In further embodiments, the cells may be MSC, osteoprogenitors, osteoblastic cells, osteocytes, chondroblastic cells, chondrocytes, adipoblastic cells, adipocytes, myoblastic cells, or myocytes. Even more preferably, the cells may be MSC, osteoprogenitors, osteoblastic cells, chondroblastic cells, or chondrocytes. Even more preferably, the cells may be MSC. Also particularly preferably, the cells may be osteoprogenitors or osteoblastic cells. In such cell types, the advantageous effect of GDF-8 on reducing immunogenicity of the cells tends to be particularly demonstrable.

Hence, in some embodiments, cells may be MSC, preferably adult human MSC. Such MSC cells may be suitably but without limitation cultured in a medium comprising serum or plasma and optionally FGF-2. In particular, FGF-2 may be included when differentiation of the MSC into cells of osteocytic lineage, e.g., osteoprogenitors or osteoblastic cells, is intended. The cells can be intended for autologous or allogeneic use, preferably, the cells can be intended for allogeneic use. In other embodiments, the cells may be osteoprogenitors or osteoblastic cells. Such osteoprogenitors or osteoblastic cells may be suitably but without limitation cultured in a medium comprising serum or plasma and optionally FGF-2. The cells can be intended for autologous or allogeneic use, preferably, the cells can be intended for allogeneic use.

The aforementioned uses may be applied to animal cells, preferably to warm-blooded animal cells. Yet more preferably, the cells intended for these uses are mammalian cells, such as human cells or non-human mammalian cells, still more preferably human cells.

In some embodiments, the present invention relates to the use of GDF-8 for reducing the immunogenicity of cells in vitro, wherein GDF-8 reduces MHC class II cell surface receptor complex on the cells and optionally reduces one or more costimulatory factors on said cells.

The recitation “reduces MHC class II cell surface receptor complex on the cells”, as used herein, refers to a reduced quantity and/or availability (e.g., availability for performing its biological activity) of MHC class II cell surface receptor complex on the cells. This reduced quantity and/or availability encompasses a decreased amount of MHC class II cell surface receptor complex on the cells and/or a decreased fraction of the cells expressing MHC class II cell surface receptor complex in a cell population. For example, on human cells MHC class II cell surface receptor complex may be an MHC class II cell surface receptor complex encoded by the HLA complex such as HLA-DR, HLA-DQ, HLA-DP, HLA-DO, or HLA-DM. Preferably, on human cells the MHC class II cell surface receptor complex may be HLA-DR. Hence, further disclosed herein is the use of GDF-8 for reducing the immunogenicity of human cells in vitro, wherein GDF-8 reduces HLA-DR on the human cells.

The recitation “reduces one or more costimulatory factors on the cells” as used herein, refers to a reduced quantity and/or availability (e.g. availability for performing their biological activity) of one or more costimulatory factors on the cells. This reduced quantity and/or availability encompasses a decreased amount of one or more costimulatory factors on the cells and/or a decreased fraction of the cells expressing one or more costimulatory factors in a cell population.

Further, without limitation, any one and all of (i) to (viii) as elaborated here below are in particular provided by this aspect of the invention:

(i) Use of GDF-8 for reducing the immunogenicity of cells in vitro;
(ii) The use as set forth in (i) above, wherein GDF-8 reduces MHC class II cell surface receptor complex on said cells and optionally reduces one or more costimulatory factors on said cells;
(iii) The use as set forth in (i) or (ii) above, wherein said cells are animal cells, preferably mammalian cells such as human cells or non-human mammalian cells;
(iv) The use as set forth in (ii) or (iii) above, wherein on human cells said MHC class II cell surface receptor is human leukocyte antigen DR (HLA-DR);
(v) The use as set forth in any of (i) to (iv) above, wherein said cells are MSC, preferably adult human MSC;
(vi) The use as set forth in any of claims (i) to (iv) above, wherein said cells are osteoprogenitors or osteoblastic cells.

In certain embodiments, the cells as specified above may be comprised in material to be administered to the subject, such as preferably in an implant or transplant (more preferably in an osseous and/or articular tissue implant or transplant (e.g., bone marrow- or bone-tissue implant or transplant)), or in a pharmaceutical formulation. By reducing the immunogenicity of the cells comprised in said materials, such as implants, transplants or pharmaceutical formulations, the risk of rejection of such therapeutically relevant products or compositions by subjects to whom they are administered can be reduced.

A further aspect of the invention relates to GDF-8 for use in reducing the immunogenicity of cells, wherein GDF-8 is to be administered in vivo. GDF-8 can be administered locally, for example, at a site of musculoskeletal lesion, for example by injection. GDF-8 can be administered alone or in combination with cells such as stem cells, preferably MSC, preferably with adult human MSC, and/or with cells such as osteoprogenitors or osteoblastic cells or with a cell population comprising osteoprogenitors and/or osteoblastic cells, preferably wherein any such cells may be human. Preferably, GDF-8 can be administered in combination with MSC, more preferably adult human MSC. Hence, further provided is GDF-8 for use in reducing the immunogenicity of cells, wherein GDF-8 is to be administered with MSC, preferably with adult human MSC, in vivo. GDF-8, optionally in combination with cells as discussed above, may further be co-administered with FGF-2. Preferably, the subject in which said administration is to be performed may be human. Preferably, the cells or cell populations to be administered may be autologous or allogeneic to said subject. GDF-8 and the respective cells or cell populations such MSC may be administered simultaneously in vivo, or may be administered sequentially in any order in vivo, for example, the respective cells or cell populations such MSC can be administered in vivo and subsequently GDF-8 can be administered in vivo, or GDF-8 can be administered in vivo and subsequently the respective cells or cell populations such MSC can be administered in vivo. Accordingly, also disclosed is use of GDF-8 for the manufacture of a medicament for reducing the immunogenicity of cells, wherein GDF-8 is to be administered in vivo; also disclosed is a method for reducing the immunogenicity of cells in a subject in need thereof, comprising administering GDF-8 to said subject.

Another aspect of the invention provides GDF-8 for use as a medicament, preferably for use in the treatment of a musculoskeletal disease including bone diseases and osteoarticular diseases. Accordingly, also provided is use of GDF-8 for the manufacture of a medicament for the treatment of musculoskeletal diseases, including bone diseases and osteoarticular diseases. Further provided is a method for treating musculoskeletal diseases, including bone diseases and osteoarticular diseases, in a subject in need of such treatment, comprising administering to said subject GDF-8.

Particularly intended is a method for treating musculoskeletal diseases in a subject in need of such treatment, comprising administering to said subject a therapeutically or prophylactically effective amount of GDF-8. When used as a medicament, GDF-8 can be administered alone or in combination with cells such as stem cells, preferably MSC, preferably with adult human MSC, and/or with cells such as osteoprogenitors or osteoblastic cells or with a cell population comprising osteoprogenitors and/or osteoblastic cells, preferably wherein any such cells may be human. Preferably, GDF-8 can be administered in combination with MSC, more preferably adult human MSC. Hence, particularly disclosed is GDF-8 for use as a medicament, preferably for use in the treatment of musculoskeletal diseases including bone diseases and osteoarticular diseases, wherein GDF-8 is to be administered in vivo together with MSC, preferably with adult human MSC. GDF-8, optionally in combination with cells as discussed above, may further be co-administered with FGF-2. Further provided is a method for treating musculoskeletal diseases, including bone diseases and osteoarticular diseases, in a subject in need of such treatment, comprising administering to said subject GDF-8 with MSC, preferably with adult human MSC. Preferably, the subject in which said administration is to be performed may be human. Preferably, the cells or cell populations to be administered may be autologous or allogeneic to said subject.

An aspect of the invention thus provides GDF-8 for use in a method of reducing the immunogenicity of cells, wherein GDF-8 is to be administered in vivo, and wherein the cells are selected from the group consisting of MSC, cells obtained by differentiation of MSC, cells of osteocytic lineage, cells of chondrocytic lineage, cells of adipocytic lineage, cells of myocytic lineage, cells of tendonocytic lineage, cells of fibroblastic lineage, and cells of stromogenic lineage. Preferably, the cells may be as dealt with in more detail in connection with in vitro uses of GDF-8 (supra). Also encompassed by this aspect is use of GDF-8 for the manufacture of a medicament for reducing the immunogenicity of cells, wherein GDF-8 is to be administered in vivo, and wherein the cells are selected from the group consisting of MSC, cells obtained by differentiation of MSC, cells of osteocytic lineage, cells of chondrocytic lineage, cells of adipocytic lineage, cells of myocytic lineage, cells of tendonocytic lineage, cells of fibroblastic lineage, and cells of stromogenic lineage; as well as a method for reducing the immunogenicity of cells in a subject in need thereof, comprising administering GDF-8 to said subject, wherein the cells are selected from the group consisting of MSC, cells obtained by differentiation of MSC, cells of osteocytic lineage, cells of chondrocytic lineage, cells of adipocytic lineage, cells of myocytic lineage, cells of tendonocytic lineage, cells of fibroblastic lineage, and cells of stromogenic lineage. Certain embodiments provide GDF-8 for use in the method of reducing the immunogenicity of cells as set forth above (or the corresponding methods or uses), wherein GDF-8 and the cells are to be administered in combination in vivo. Such embodiments advantageously allow to reduce the immunogenicity and risk of rejection of the cells that are administered to a subject. While these effects of GDF-8 may be of value in cell therapy methods employing cells that are either autologous, allogeneic or even xenogeneic to said subject, more typically autologous or allogeneic, the effects may particularly facilitate administration of allogeneic cells, since reducing the immunogenicity of allogeneic cells is expected to considerably lower risk of their rejection by the subject, and possibly allow to avoid or diminish immunosuppressive therapy that subjects typically receive when give allogeneic cell material. Accordingly, some embodiments provide GDF-8 for use in the method of reducing the immunogenicity of cells as set forth above (or the corresponding methods or uses), wherein the cells are allogeneic to a subject to whom they are to be administered.

It shall be understood that GDF-8 and the cells may be administered simultaneously in vivo, or may be administered sequentially in any order in vivo, for example, the cells can be administered in vivo and subsequently GDF-8 can be administered in vivo, or GDF-8 can be administered in vivo and subsequently the cells can be administered in vivo. In an example, GDF-8 administration may be prescribed in a subject who previously received the cells, when signs of immune reaction against the cells or rejection of the cells are detected in the subject. In a further example, GDF-8 administration may be prescribed in a subject who previously received, is receiving, or will in the future receive the cells, when there is an increased likelihood that the subject will display immune reaction against the cells or rejection of the cells (e.g., poor HLA match). In a yet further example, GDF-8 administration may be prescribed in a subject who previously received, is receiving, or will in the future receive the cells, irrespective of any actual observation or expectedly increased likelihood of immune reaction against the cells or rejection of the cells.

In certain embodiments, GDF-8 may be administered systemically, regardless of whether the cells are administered locally or systemically. In other embodiments, GDF-8 can be administered locally. For example, when the cells are administered locally (e.g., intra- or peri-osseously or intra- or peri-articularly, such as where the cells are intended for bone or joint tissue repair), GDF-8 can be preferably also administered locally, more specifically in proximity to the cells (e.g., also intra- or peri-osseously or intra- or peri-articularly, as the case may be). Suitably, GDF-8 may be formulated in a sustained-release formulation at the site of its administration, to prolong its effects on the cells.

In certain embodiments, the cells may be comprised in material administered to the subject, such as preferably in an implant or transplant (more preferably in an osseous and/or articular tissue implant or transplant (e.g., bone marrow- or bone-tissue implant or transplant)), or in a pharmaceutical formulation. By reducing the immunogenicity of the cells comprised in said materials, such as implants, transplants or pharmaceutical formulations, the risk of rejection of such therapeutically relevant products or compositions by subjects to whom they are administered can be reduced.

A further aspect of the invention thus provides combination of GDF-8 and cells for use as a medicament, preferably for use in the treatment of (i.e., for use in a method of treating) a musculoskeletal disease, more preferably wherein the musculoskeletal disease is a bone disease or an osteoarticular disease, wherein the cells are selected from the group consisting of MSC, cells obtained by differentiation of MSC, cells of osteocytic lineage, cells of chondrocytic lineage, cells of adipocytic lineage, cells of myocytic lineage, cells of tendonocytic lineage, cells of fibroblastic lineage, and cells of stromogenic lineage. Also encompassed by this aspect is use of combination of GDF-8 and cells for the manufacture of a medicament for the treatment of a musculoskeletal disease, more preferably wherein the musculoskeletal disease is a bone disease or an osteoarticular disease, wherein the cells are selected from the group consisting of MSC, cells obtained by differentiation of MSC, cells of osteocytic lineage, cells of chondrocytic lineage, cells of adipocytic lineage, cells of myocytic lineage, cells of tendonocytic lineage, cells of fibroblastic lineage, and cells of stromogenic lineage; as well as a method for treating a musculoskeletal disease, more preferably wherein the musculoskeletal disease is a bone disease or an osteoarticular disease, in a subject in need of said treatment, comprising administering combination of GDF-8 and cells (in particular a therapeutically or prophylactically effective amount of the combination) to said subject, wherein the cells are selected from the group consisting of MSC, cells obtained by differentiation of MSC, cells of osteocytic lineage, cells of chondrocytic lineage, cells of adipocytic lineage, cells of myocytic lineage, cells of tendonocytic lineage, cells of fibroblastic lineage, and cells of stromogenic lineage. Preferably, the cells may be as dealt with in more detail in connection with in vitro uses of GDF-8 (supra). Certain embodiments provide combination of GDF-8 and cells for use in the treatment of a musculoskeletal disease (or the corresponding methods or uses), preferably wherein the musculoskeletal disease is a bone disease or an osteoarticular disease, wherein the cells are selected from the group consisting of MSC, cells of osteocytic lineage, cells of chondrocytic lineage, cells of myocytic lineage, and cells of tendonocytic lineage, more preferably wherein the cells are MSC, osteoprogenitors, osteoblastic cells, osteocytes, chondroblastic cells, chondrocytes, myoblastic cells, or myocytes, even more preferably wherein the cells are MSC, osteoprogenitors, osteoblastic cells, chondroblastic cells, or chondrocytes, yet more preferably wherein the cells are MSC or wherein the cells are osteoprogenitors or osteoblastic cells.

The combinations of GDF-8 and cells as set forth in the previous paragraph may advantageously allow to reduce the immunogenicity and risk of rejection of the cells administered to a subject as a medicament, preferably for the purpose of treating the musculoskeletal disease. While these effects of GDF-8 in the combination may be of value in cell therapy methods employing cells that are either autologous, allogeneic or even xenogeneic to said subject, more typically autologous or allogeneic, the effects may particularly facilitate administration of allogeneic cells, since reducing the immunogenicity of allogeneic cells is expected to considerably lower risk of their rejection by the subject, and possibly allow to avoid or diminish immunosuppressive therapy that subjects typically receive when give allogeneic cell material. Accordingly, certain embodiments provide the combination of GDF-8 and cells for use as a medicament, preferably for use in the treatment of a musculoskeletal disease, as set forth above (or the corresponding methods or uses), wherein the cells are allogeneic to a subject to whom they are to be administered.

It shall be understood that GDF-8 and the cells comprised in the combination may be administered simultaneously in vivo, or may be administered sequentially in any order in vivo, for example, the cells can be administered in vivo and subsequently GDF-8 can be administered in vivo, or GDF-8 can be administered in vivo and subsequently the cells can be administered in vivo. In an example, GDF-8 administration may be prescribed in a subject who previously received the cells, when signs of immune reaction against the cells or rejection of the cells are detected in the subject. In a further example, GDF-8 administration may be prescribed in a subject who previously received, is receiving, or will in the future receive the cells, when there is an increased likelihood that the subject will display immune reaction against the cells or rejection of the cells (e.g., poor HLA match). In a yet further example, GDF-8 administration may be prescribed in a subject who previously received, is receiving, or will in the future receive the cells, irrespective of any actual observation or expectedly increased likelihood of immune reaction against the cells or rejection of the cells.

In certain embodiments, GDF-8 may be administered systemically, regardless of whether the cells are administered locally or systemically. In other embodiments, GDF-8 can be administered locally. For example, when the cells are administered locally (e.g., intra- or peri-osseously or intra- or peri-articularly, such as where the cells are intended for bone or joint tissue repair), GDF-8 can be preferably also administered locally, more specifically in proximity to the cells (e.g., also intra- or peri-osseously or intra- or peri-articularly, as the case may be). Suitably, GDF-8 may be formulated in a sustained-release formulation at the site of its administration, to prolong its effects on the cells.

In certain embodiments, the cells in the combination of GDF-8 and cells may be comprised in material administered to the subject, such as preferably in an implant or transplant (more preferably in an osseous and/or articular tissue implant or transplant (e.g., bone marrow- or bone-tissue implant or transplant)), or in a pharmaceutical formulation. By reducing the immunogenicity of the cells comprised in said materials, such as implants, transplants or pharmaceutical formulations, the risk of rejection of such therapeutically relevant products or compositions by subjects to whom they are administered can be reduced.

Following from the observation of the ability of GDF-8 to regulate immunogenicity and immune rejection, a further aspect of the invention provides GDF-8 for use in reducing the risk of rejection by a subject of a material administered, implanted or transplanted into the subject. Also encompassed by this aspect is use of GDF-8 for the manufacture of a medicament for reducing the risk of rejection by a subject of a material administered, implanted or transplanted into the subject; as well as a method for reducing the risk of rejection by a subject of a material administered, implanted or transplanted into the subject, comprising administering GDF-8 to said subject. In the context of this and the foregoing aspects and embodiments, the reference to a material (to be) administered to the subject is intended to broadly encompass any materials, which may be of benefit when administered to the subject, for example but without limitation, which may be of therapeutic or prophylactic benefit in the subject (e.g., which may be useful to treat a musculoskeletal disease, more preferably wherein the musculoskeletal disease is a bone disease or an osteoarticular disease, in the subject). Certain embodiments provide GDF-8 for the stated use (or the corresponding methods or uses), wherein the material comprises osseous and/or articular tissue (e.g., bone marrow- or bone-tissue).

In certain embodiments, the material may comprise cells selected from the group consisting of MSC, cells obtained by differentiation of MSC, cells of osteocytic lineage, cells of chondrocytic lineage, cells of adipocytic lineage, cells of myocytic lineage, cells of tendonocytic lineage, cells of fibroblastic lineage, and cells of stromogenic lineage. Preferably, the cells may be as dealt with in more detail in connection with in vitro uses of GDF-8 (supra). Preferably, where the material is intended in the treatment of a musculoskeletal disease, preferably wherein the musculoskeletal disease is a bone disease or an osteoarticular disease, and the material contains cells, the cells may be selected from the group consisting of MSC, cells of osteocytic lineage, cells of chondrocytic lineage, cells of myocytic lineage, and cells of tendonocytic lineage, more preferably selected from the group consisting of MSC, osteoprogenitors, osteoblastic cells, osteocytes, chondroblastic cells, chondrocytes, myoblastic cells, or myocytes, even more preferably selected from the group consisting of MSC, osteoprogenitors, osteoblastic cells, chondroblastic cells, or chondrocytes, yet more preferably selected from the group consisting of MSC or selected from the group consisting of osteoprogenitors or osteoblastic cells.

The effects of GDF-8 on diminishing the risk of rejection by a subject of the material may be particularly pronounced in certain embodiments, when the material bears a significant risk of rejection, for example, when the material contains one or more components, e.g., tissues, cells, biomolecules or other substances, allogeneic or even xenogeneic to the subject, more typically allogeneic.

It shall be understood that GDF-8 and the material may be administered simultaneously in vivo, or may be administered sequentially in any order in vivo, for example, the material can be administered in vivo and subsequently GDF-8 can be administered in vivo, or GDF-8 can be administered in vivo and subsequently the material can be administered in vivo. In an example, GDF-8 administration may be prescribed in a subject who previously received the material, when signs of rejection of the material are detected in the subject. In a further example, GDF-8 administration may be prescribed in a subject who previously received, is receiving, or will in the future receive the material, when there is an increased likelihood that the subject will display rejection of the material (e.g., poor HLA match). In a yet further example, GDF-8 administration may be prescribed in a subject who previously received, is receiving, or will in the future receive the material, irrespective of any actual observation or expectedly increased likelihood of rejection of the material.

In certain embodiments, GDF-8 may be administered systemically, regardless of whether the material is administered locally or systemically. In other embodiments, GDF-8 can be administered locally. For example, when the material is administered locally (e.g., intra- or peri-osseously or intra- or peri-articularly, such as where the material is intended for bone or joint tissue repair), GDF-8 can be preferably also administered locally, more specifically in proximity to the material (e.g., also intra- or peri-osseously or intra- or peri-articularly, as the case may be). Suitably, GDF-8 may be formulated in a sustained-release formulation at the site of its administration, to prolong its effects on the material.

Accordingly, a further aspect of the invention provides a pharmaceutical composition comprising a material to be administered, implanted or transplanted into a subject and GDF-8, and optionally further comprising one or more pharmaceutically acceptable excipients. Preferably provided is a pharmaceutical composition comprising cells selected from the group consisting of MSC, cells obtained by differentiation of MSC, cells of osteocytic lineage, cells of chondrocytic lineage, cells of adipocytic lineage, cells of myocytic lineage, cells of tendonocytic lineage, cells of fibroblastic lineage, and cells of stromogenic lineage, and GDF-8, and optionally further comprising one or more pharmaceutically acceptable excipients (in other words, it can be said, that the material to be administered, implanted or transplanted into the subject comprises the recited cells). Preferably, the cells may be as dealt with in more detail in connection with in vitro uses of GDF-8 (supra). Also preferably provided is a pharmaceutical composition comprising osseous and/or articular tissue (e.g., bone marrow- or bone-tissue) and GDF-8, and optionally further comprising one or more pharmaceutically acceptable excipients (in other words, it can be said, that the material to be administered, implanted or transplanted into the subject comprises the recited osseous and/or articular tissue).

Throughout the present specification, the products, methods and uses embodying the principles of the invention may preferably employ animal cells, more preferably warm-blooded animal cells, yet more preferably mammalian cells, such as human cells or non-human mammalian cells, still more preferably human cells.

Further, throughout the present specification, the products, methods and uses embodying the principles of the invention may be applied to animal subjects, more preferably warm-blooded animal subjects, yet more preferably mammalian subjects, such as human subjects or non-human mammalian subjects, still more preferably human subjects.

It shall also be appreciated that cells, tissues or other materials originating from a certain species (e.g., a given mammalian species, or human) would typically be administered to a subject from the same species, i.e., autologous or allogeneic administration.

The present inventors have now further found a method for differentiating mesenchymal stem cells (MSC) addressing one or more of the above-mentioned problems of the prior art.

Hence, an additional aspect of the present invention relates to a method for differentiating adult MSC in vitro or ex vivo into osteoprogenitors or osteoblastic cells or a cell population comprising osteoprogenitors and/or osteoblastic cells, the method comprising the step of culturing said MSC in a medium comprising plasma or serum, growth and differentiation factor 8 (GDF-8) and fibroblast growth factor 2 (FGF-2). Methods applying the principles of the present invention advantageously allow to obtain osteoprogenitors or osteoblastic cells or cell populations comprising osteoprogenitors and/or osteoblastic cells with decreased immunogenicity.

For example, the present methods provide osteoprogenitors, or osteoblastic cells or cell populations comprising osteoprogenitors and/or osteoblastic cells with decreased expression of MHC class II cell surface receptor, for example with decreased expression of HLA-DR. Such decreased immunogenicity advantageously allows cell transplantation for instance to allogeneic subjects.

In addition, the inventors found that the methods of the present invention stimulate cell proliferation. Such methods thus have the advantage to generate cells suitable for transplantation in an amount which is satisfactory or improved for cell transplantation. This also permits to reduce the amount of tissue which needs to be taken from a subject to obtain the starting MSC.

Hence, the methods of the present invention advantageously provide for osteoprogenitors or osteoblastic cells or cell populations comprising osteoprogenitors and/or osteoblastic cells with an improved transplantation potential.

The inventors verified that the methods of the present invention maintain the desired osteoblastic phenotype of the obtained osteoprogenitors or osteoblastic cells or cell populations comprising osteoprogenitors and/or osteoblastic cells. This is unexpected inter alia because an increase of osteogenic differentiation of MSC has been previously documented in GDF-8 deficient mice (Hamrick et al., Bone, 2007, vol. 40(6), 1544-53).

In one embodiment, the method of the present invention can comprise the steps of: (a) allowing cells recovered from a biological sample of a subject and comprising MSC to adhere to a substrate surface; and (b) culturing the adherent cells in a medium comprising plasma or serum, GDF-8 and FGF-2, such as to allow for differentiating MSC in vitro or ex vivo into osteoprogenitors or osteoblastic cells or the cell population comprising osteoprogenitors and/or osteoblastic cells.

In certain methods embodying the principles of the present invention, the cells may be cultured in the medium as defined in step (b) for a period of between about 10 and about 18 days. Such period allows to produce an amount of osteoprogenitors or osteoblastic cells or cell populations comprising osteoprogenitors and/or osteoblastic cells particularly satisfactory for cell transplantation.

Some methods according to the present invention may further include step (c) passaging (e.g., passaging one or more times) and further culturing the osteoprogenitors or osteoblastic cells or cell populations comprising osteoprogenitors and/or osteoblastic cells from step (b) in the medium as defined in (b). For example but without limitation, the cells may be cultured in step (c) for a period of between about 3 and about 18 days, to allow to produce an amount of osteoprogenitors or osteoblastic cells or cell populations comprising osteoprogenitors and/or osteoblastic cells particularly satisfactory for cell therapy

In further embodiments, the method of the present invention can comprise the steps of: (a) allowing cells recovered from a biological sample of a subject and comprising MSC to adhere to a substrate surface; (b′) culturing the adherent cells in a medium comprising plasma or serum and FGF-2; and (b″) further culturing the adherent cells in a medium comprising plasma or serum, GDF-8 and FGF-2, such as to allow for differentiating MSC in vitro or ex vivo into the osteoprogenitors or osteoblastic cells or the cell population comprising osteoprogenitors and/or osteoblastic cells. In some embodiments, the method of the present invention can further include between step (b′) and (b″), the step (c′) of passaging and allowing cells to adhere to a substrate surface.

It shall be appreciated that methods embodying the principles of the present invention may further comprise one or more steps of passaging the cells, i.e. passages, such as one, two, three, four, or more passages. In preferred embodiments, the method may further comprise one, two or three passages, more preferably, one or two passages, even more preferably, one passage.

In this respect, the terms “primary culture”, “secondary culture” and “tertiary culture”, as used herein, refer to cells recovered from a biological sample of a subject and comprising MSC which during the present method have not undergone any passage, have undergone one passage or have undergone two passages respectively. In the present methods, culturing MSC in a medium comprising plasma or serum, GDF-8 and FGF-2, may be typically but without limitation performed from primary culture, for example, from the start (or beginning) of primary culture or from within primary culture; from secondary culture, for example, from the start (or beginning) of secondary culture or from within secondary culture; or from tertiary culture, for example, from the start (or beginning) of tertiary culture or from within tertiary culture. Preferably, culturing MSC in a medium comprising plasma or serum, GDF-8 and FGF-2 may be performed from primary culture, for example, from the start of primary culture or from within primary culture, preferably, from the start of primary culture.

As illustrated in the examples, culturing MSC in a medium comprising plasma or serum, GDF-8 and FGF-2 from the start of primary culture allows to obtain osteoprogenitors or osteoblastic cells or cell populations comprising osteoprogenitors and/or osteoblastic cells with particularly reduced immunogenicity, more specifically reduced MHC class II cell surface receptor expression. As further illustrated in the examples, culturing MSC in a medium comprising plasma or serum, GDF-8 and FGF-2 from the start of primary culture also particularly stimulates cell proliferation. Such methods illustrating the present invention are therefore particularly advantageous because they achieve a greater degree of cell expansion and can produce osteoprogenitors or osteoblastic cells or cell populations comprising osteoprogenitors and/or osteoblastic cells with reduced immunogenicity particularly suitable for transplantation, such as for example, cells causing less rejection in allogeneic subjects.

In further embodiments, the methods as taught herein may comprise the step of contacting (e.g., bringing together or admixing) the resultant osteoprogenitors or osteoblastic cells or the cell population comprising osteoprogenitors and/or osteoblastic cells with a component having osteogenic, osteo-inductive and/or osteo-conductive properties. Such step may particularly allow for the preparation of pharmaceutical compositions suitable for transplantation, for instance to an allogeneic subject.

The methods and uses as intended herein may be particularly preferably applied to animal cells, preferably to warm-blooded animal cells, more preferably to mammalian cells, such as human cells or non-human mammalian cells, and most preferably to human cells. Another aspect of the present invention provides osteoprogenitors or osteoblastic cells or a cell population comprising osteoprogenitors and/or osteoblastic cells obtainable by any of the present methods. Particularly disclosed are osteoprogenitors or osteoblastic cells or cell populations comprising osteoprogenitors and/or osteoblastic cells obtainable by a method as defined above for differentiating adult MSC in vitro or ex vivo, comprising the step of culturing the MSC in a medium comprising plasma or serum, GDF-8 and FGF-2. It shall be appreciated that the present methods may generally produce cell populations comprising a substantial portion, e.g., a majority of, osteoprogenitors or osteoblastic cells. Such cell populations may further include other cell types.

Also provided in an aspect of the invention is a pharmaceutical composition comprising the osteoprogenitors or osteoblastic cells or the cell population comprising osteoprogenitors and/or osteoblastic cells as taught herein and further suitably comprising an excipient, preferably wherein at least one of said excipients is a component with osteogenic, osteo-inductive and/or osteo-conductive properties.

A further aspect of the invention provides the osteoprogenitors or osteoblastic cells or the cell population comprising osteoprogenitors and/or osteoblastic cells as taught herein or the pharmaceutical composition as defined above for use as a medicament, preferably for use in the treatment (including throughout the present specification therapeutic and/or preventative measures) of a musculoskeletal disease. Preferably, said musculoskeletal disease may be a bone disease or an osteoarticular disease. Hence, preferably intended are osteoprogenitors or osteoblastic cells or a cell population comprising osteoprogenitors and/or osteoblastic cells, obtainable by the method of the present invention, for use in the treatment of musculoskeletal diseases, such as without limitation bone diseases and/or osteoarticular diseases.

The use of said osteoprogenitors or osteoblastic cells or the cell population comprising osteoprogenitors and/or osteoblastic cells in the treatment of musculoskeletal diseases is advantageous for example because they allow transplantation to an allogeneic subject due to the reduced immunogenicity of such cells or cell populations.

Also provided according to the present invention is the use of the osteoprogenitors or osteoblastic cells or the cell population comprising osteoprogenitors and/or osteoblastic cells as taught herein for the manufacture of a medicament for the treatment of musculoskeletal diseases, including among others bone diseases and osteoarticular diseases. Thus, particularly intended is use of the osteoprogenitors or osteoblastic cells or the cell population comprising osteoprogenitors and/or osteoblastic cells obtainable by the method of the present invention for the manufacture of a medicament for the treatment of musculoskeletal diseases, such as without limitation bone diseases and/or osteoarticular diseases.

Further provided according to the present invention is a method for treating musculoskeletal diseases, including among others bone diseases and osteoarticular diseases, in a subject in need of such treatment, comprising administering to said subject the osteoprogenitors or osteoblastic cells or the cell population comprising osteoprogenitors and/or osteoblastic cells as taught herein or the pharmaceutical compositions as defined above. Particularly intended is a method for treating musculoskeletal diseases in a subject in need of such treatment, comprising administering to said subject a therapeutically or prophylactically effective amount of the osteoprogenitors or osteoblastic cells or the cell population comprising osteoprogenitors and/or osteoblastic cells obtainable by the method of the present invention.

Hence, without limitation, any one and all of (i′) to (viii′) as elaborated here below are provided by this aspect of the invention:

(i′) Method for differentiating adult mesenchymal stem cells (MSC) in vitro or ex vivo into osteoprogenitors or osteoblastic cells or a cell population comprising osteoprogenitors and/or osteoblastic cells, said method comprising the step of culturing said MSC in a medium comprising plasma or serum, growth and differentiation factor 8 (GDF-8) and fibroblast growth factor 2 (FGF-2);
(ii′) The method as set forth in (i′) above, comprising the steps of:
(a) allowing cells recovered from a biological sample of a subject and comprising MSC to adhere to a substrate surface; and
(b) culturing adherent cells in a medium comprising plasma or serum, GDF-8 and FGF-2, such as to allow for differentiating MSC in vitro or ex vivo into the osteoprogenitors or osteoblastic cells or the cell population comprising osteoprogenitors and/or osteoblastic cells;
   (iii′) The method as set forth in (ii′) above, further including step (c) passaging and further culturing the osteoprogenitors or osteoblastic cells or the cell population comprising osteoprogenitors and/or osteoblastic cells from step (b) in the medium as defined in (b);
   (iv′) The method as set forth in (i′) above, comprising the steps of:
(a) allowing cells recovered from a biological sample of a subject and comprising MSC to adhere to a substrate surface;
(b′) culturing adherent cells in a medium comprising plasma or serum and FGF-2; and
(b″) further culturing adherent cells in a medium comprising plasma or serum, GDF-8 and FGF-2, such as to allow for differentiating MSC in vitro or ex vivo into osteoprogenitors or osteoblastic cells or the cell population comprising osteoprogenitors and/or osteoblastic cells;
   (v′) The method as set forth in (iv′) above, further including between step (b′) and (b″), the step (c′) of passaging and allowing cells to adhere to a substrate surface.
   (vi′) The method as set forth in any one of (i′) to (v′) above, further comprising the step of contacting said osteoprogenitors or osteoblastic cells or the cell population comprising osteoprogenitors and/or osteoblastic cells with a component with osteogenic, osteo-inductive and/or osteo-conductive properties;
   (vii′) Osteoprogenitors or osteoblastic cells or a cell population comprising osteoprogenitors and/or osteoblastic cells obtainable by the method of any of (i′) to (vi′) above or a pharmaceutical composition comprising the same;
   (viii′) The osteoprogenitors or osteoblastic cells or the cell population comprising osteoprogenitors and/or osteoblastic cells as defined in (vii′) above or the pharmaceutical composition as defined in (vii) above for use as a medicament, preferably for use in the treatment of a musculoskeletal disease, more preferably wherein the musculoskeletal disease is a bone disease or an osteoarticular disease.

The above and further aspects and preferred embodiments of the invention are described in the following sections and in the appended claims. The subject-matter of appended claims is hereby specifically incorporated in this specification.

### BRIEF DESCRIPTION OF DRAWINGS

FIG. 1 represents a graph illustrating the mean global yield (%) of cells cultured in a medium comprising (1) serum and FGF-2 (control), (2) serum, FGF-2 and TGF-beta 1 and (3) serum, FGF-2 and GDF-8.

FIG. 2 represents a graph illustrating the HLA-DR expression (%) of cells cultured from the start of primary culture in a medium comprising (1) serum and FGF-2 (control), (2) serum, FGF-2 and TGF-beta 1 or (3) serum, FGF-2 and GDF-8.

FIG. 3 represents a graph illustrating the expression (%) of alkaline phosphatase (ALP) of cells cultured from the start of primary culture in a medium comprising (1) serum and FGF-2, (2) serum, FGF-2 and TGF-beta 1 or (3) serum, FGF-2 and GDF-8.

FIG. 4 represents a graph illustrating the concentration (in pg/ml) of (4) IL6, (5) VEGF, (6) decorin and (7) osteoprotegerin, respectively, in the supernatant of cells cultured from the start of primary culture in a medium comprising (1) serum and FGF-2 (control), (2) serum, FGF-2 and TGF-beta 1 or (3) serum, FGF-2 and GDF-8.

FIG. 5 represents an assay illustrating the mineralization after secondary culture of cells cultured from the start of primary culture in a medium comprising serum and FGF-2 (FGF-2); serum, FGF-2 and TGF-beta 1 (TGF-beta 1); or serum, FGF-2 and GDF-8 (GDF-8). C: control medium, M: osteogenic medium.

FIG. 6 represents a graph illustrating the percentage of positive cells (5) before culturing the cells for 4 days and after culturing the cells from tertiary culture for 4 days in a medium comprising (6) serum and FGF-2 (control), (7) serum, FGF-2 and TGF-beta 1 or (8) serum, FGF-2 and GDF-8. 1: CD45, 2: CD 105, 3: HLA-DR.

FIG. 7 represents a graph illustrating the expression of HLA-DR (%) of cells after culturing the cells from tertiary culture for 6 days in a medium comprising (1) serum and FGF-2 (control), (2) serum, FGF-2 and 1 ng/ml TGF-beta 1 (3) serum, FGF-2 and 50 ng/ml GDF-8, (4) serum, FGF-2 and 100 ng/ml GDF-8, or (5) serum, FGF-2 and 200 ng/ml GDF-8.

FIG. 8 represents a graph illustrating the global yield of primary and secondary culture (%) of cells from one batch cultured from the start of primary culture in a medium comprising (1) serum, FGF-2 and GDF-8 and (2) serum and GDF-8.

FIG. 9 represents a graph illustrating the expression (%) of alkaline phosphatase (ALP) of cells from one batch cultured from the start of primary culture in a medium comprising (1) serum and FGF-2, (2) serum, FGF-2 and GDF-8 or (3) serum and GDF-8.

FIG. 10 represents a graph illustrating the HLA-DR expression (%) of cells from one batch cultured from the start of primary culture in a medium comprising (1) serum and FGF-2, (2) serum, FGF-2 and GDF-8 or (3) serum and GDF-8.

### DETAILED DESCRIPTION OF THE INVENTION

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms also encompass “consisting of” and “consisting essentially of”.

The recitation of numerical ranges by endpoints includes all numbers and fractions subsumed within the respective ranges, as well as the recited endpoints.

The term “about” as used herein when referring to a measurable value such as a parameter, an amount, a temporal duration, and the like, is meant to encompass variations of and from the specified value, in particular variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

Whereas the term “one or more”, such as one or more members of a group of members, is clear per se, by means of further exemplification, the term encompasses inter alia a reference to any one of said members, or to any two or more of said members, such as, e.g., any ≧3, ≧4, ≧5, ≧6 or ≧7 etc. of said members, and up to all said members.

All documents cited in the present specification are hereby incorporated by reference in their entirety.

Unless otherwise specified, all terms used in disclosing the invention, including technical and scientific terms, have the meaning as commonly understood by one of ordinary skill in the art to which this invention belongs. By means of further guidance, term definitions may be included to better appreciate the teaching of the present invention.

General techniques in cell culture and media uses are outlined inter alia in Large Scale Mammalian Cell Culture (Hu et al. 1997. Curr Opin Biotechnol 8: 148); Serum-free Media (K. Kitano. 1991. Biotechnology 17: 73); or Large Scale Mammalian Cell Culture (Curr Opin Biotechnol 2: 375, 1991).

As noted, the present inventors found according to an aspect of the present invention a method for differentiating adult mesenchymal stem cells (MSC) in vitro or ex vivo into osteoprogenitors or osteoblastic cells or a cell population comprising osteoprogenitors and/or osteoblastic cells, comprising the step of culturing said MSC in a medium comprising plasma or serum, growth and differentiation factor 8 (GDF-8) and fibroblast growth factor 2 (FGF-2).

Expanding on these findings, the inventors further recognized the ability of GDF-8 to reduce the immunogenicity of cells, and in certain aspects of the invention provide uses, methods and products which employ GDF-8 to reduce the immunogenicity of cells in vitro or in vivo. In related aspects of the invention, the inventors contemplate uses, methods and products which employ GDF-8 to reduce the risk of rejection by a subject of a material administered, implanted or transplanted into the subject.

The term “immunogenicity”, as used herein, refers to the ability of a particular substance, such as a cell to provoke an immune response in the body of a human or animal. This ability depends on immunogens such as an antigen or an epitope presented on the cells. Such an immunogen may be for instance but without limitation any major histocompatibility complex (MHC) class II cell surface receptor complex, such as any human leukocyte antigen (HLA), preferably HLA-DR. The term “HLA-DR”, as used herein, is well-known per se and particularly refers to a MHC class II cell surface receptor complex encoded by the human leukocyte antigen complex on chromosome 6 region 6p21.31. The immunogenicity may further depend on costimulatory factors to provide a costimulatory signal in the immune response. Such a costimulatory factor may be for instance but without limitation one or more of cluster of differentiation 80 (CD80 or B7-1) or cluster of differentiation 86 (CD86 or B7-2).

Accordingly, in a further aspect, the invention provides the use of GDF-8 for reducing the immunogenicity of cells in vitro. The reduced immunogenicity can comprise reduced MHC class II cell surface receptor complex compared with respective reference value(s) representing MHC class II cell surface receptor complex in cells cultured without GDF-8. For instance, on human cells the reduced immunogenicity can comprise reduced HLA MHC class II cell surface receptor complex compared with respective reference value(s) representing HLA MHC class II cell surface receptor complex in cells cultured without GDF-8. Preferably, the reduced immunogenicity comprises reduced HLA-DR compared with respective reference value(s) representing HLA-DR in cells cultured without GDF-8. The terms “reducing”, “decreasing”, “diminishing” or “lowering” can be used interchangeably herein.

The recitations “reduced MHC class II cell surface receptor complex”, “reduced HLA MHC class II cell surface receptor complex” or “reduced HLA-DR” refer to a reduced quantity and/or availability (e.g. availability for performing its biological activity) on the cells of MHC class II cell surface receptor complex, HLA MHC class II cell surface receptor complex or HLA-DR respectively.

The recitation “reduces HLA-DR on the cells”, as used herein, refers to a reduced quantity and/or availability (e.g. availability for performing its biological activity) of HLA-DR on the cells. This reduced quantity and/or availability encompasses a decreased amount of HLA-DR on the cells and/or a decreased fraction of the cells expressing HLA-DR in a cell population.

For example and without limitation, where the reduced quantity and/or availability encompasses a decreased fraction of the cells expressing HLA-DR in a cell population compared with respective reference value(s) representing HLA-DR in cells cultured without GDF-8, less than 25% of the cells, preferably less than 20% of the cells and even more preferably less than 15% of the cells may express HLA-DR.

A further aspect of the invention relates to GDF-8 for use in reducing the immunogenicity of cells, wherein GDF-8 is to be administered in vivo. GDF-8 can be administered locally, for example, at a site of musculoskeletal lesion, for example by injection. GDF-8 can be administered alone or in combination with cells such as stem cells, preferably MSC, preferably with adult human MSC, and/or with cells such as osteoprogenitors or osteoblastic cells or with a cell population comprising osteoprogenitors and/or osteoblastic cells, preferably wherein any such cells may be human. Preferably, GDF-8 can be administered in combination with MSC, more preferably adult human MSC. Hence, further provided is GDF-8 for use in reducing the immunogenicity of cells, wherein GDF-8 is to be administered with MSC, preferably with adult human MSC, in vivo. GDF-8, optionally in combination with cells as discussed above, may further be co-administered with FGF-2. Preferably, the subject in which said administration is to be performed may be human.

Preferably, the cells or cell populations to be administered may be autologous or allogeneic to said subject. GDF-8 and the respective cells or cell populations such MSC may be administered simultaneously in vivo, or may be administered sequentially in any order in vivo, for example, the respective cells or cell populations such MSC can be administered in vivo and subsequently GDF-8 can be administered in vivo, or GDF-8 can be administered in vivo and subsequently the respective cells or cell populations such MSC can be administered in vivo. Accordingly, also disclosed is use of GDF-8 for the manufacture of a medicament for reducing the immunogenicity of cells, wherein GDF-8 is to be administered in vivo; also disclosed is a method for reducing the immunogenicity of cells in a subject in need thereof, comprising administering GDF-8 to said subject.

Another aspect of the invention provides GDF-8 for use as a medicament, preferably for use in the treatment of a musculoskeletal disease including bone diseases and osteoarticular diseases. Accordingly, also provided is use of GDF-8 for the manufacture of a medicament for the treatment of musculoskeletal diseases, including bone diseases and osteoarticular diseases. Further provided is a method for treating musculoskeletal diseases, including bone diseases and osteoarticular diseases, in a subject in need of such treatment, comprising administering to said subject GDF-8. Particularly intended is a method for treating musculoskeletal diseases in a subject in need of such treatment, comprising administering to said subject a therapeutically or prophylactically effective amount of GDF-8. When used as a medicament, GDF-8 can be administered alone or in combination with cells such as stem cells, preferably MSC, preferably with adult human MSC, and/or with cells such as osteoprogenitors or osteoblastic cells or with a cell population comprising osteoprogenitors and/or osteoblastic cells, preferably wherein any such cells may be human. Preferably, GDF-8 can be administered in combination with MSC, more preferably adult human MSC. Hence, particularly disclosed is GDF-8 for use as a medicament, preferably for use in the treatment of musculoskeletal diseases including bone diseases and osteoarticular diseases, wherein GDF-8 is to be administered in vivo together with MSC, preferably with adult human MSC. GDF-8, optionally in combination with cells as discussed above, may further be co-administered with FGF-2. Further provided is a method for treating musculoskeletal diseases, including bone diseases and osteoarticular diseases, in a subject in need of such treatment, comprising administering to said subject GDF-8 with MSC, preferably with adult human MSC. Preferably, the subject in which said administration is to be performed may be human. Preferably, the cells or cell populations to be administered may be autologous or allogeneic to said subject.

As mentioned above, the present invention relates in an aspect to a method for differentiating adult mesenchymal stem cells (MSC) in vitro or ex vivo into osteoprogenitors or osteoblastic cells or a cell population comprising osteoprogenitors and/or osteoblastic cells. The invention further relates to applications of GDF-8 in vitro or in vivo for reducing the immunogenicity of cells, particularly wherein the cells are selected from the group consisting of MSC, cells obtained by differentiation of MSC, cells of osteocytic lineage, cells of chondrocytic lineage, cells of adipocytic lineage, cells of myocytic lineage, cells of tendonocytic lineage, cells of fibroblastic lineage, and cells of stromogenic lineage.

The recitation “a cell population comprising osteoprogenitors and/or osteoblastic cells”, as used herein, refers to a cell population comprising any one or both recited cell types and optionally further containing other, non-recited, cell types.

As used herein, “osteoprogenitors” may particularly comprise early and late osteoprogenitors. “Osteoblastic cells” may particularly encompass pre-osteoblasts, osteoblasts and osteocytes. All these terms are well-known per se and as used herein may typically refer to cells having an osteogenic phenotype, and that can contribute to, or are capable of developing to cells which can contribute to, the formation of bone material or bone matrix. In particular, the present methods result in cells and cell populations which are advantageously useful for transplantation or for the treatment of musculoskeletal diseases for instance for restoring bone formation in therapeutic settings. Consequently, the terms “osteoprogenitors” (including early and late osteoprogenitors) and “osteoblastic cells” (including pre-osteoblasts, osteoblasts and osteocytes) should be construed as wishing to encompass any such useful cells of the osteogenic lineage resulting from the methods applying the principles of the present invention. Useful cells of the osteogenic lineage may thus encompass cells at any stage of osteogenic differentiation towards mature, bone-forming cells.

By means of further guidance and not limitation, osteoprogenitors and osteoblastic cells, as well as cell populations comprising osteoprogenitors and/or osteoblastic cells may display the following characteristics:

a) the cells comprise expression of Runx2, a multifunctional transcription factor that regulates osteoblast differentiation and the expression of many extracellular matrix protein genes during osteoblast differentiation;
b) the cells comprise expression of at least one of the following: alkaline phosphatase (ALP), more specifically ALP of the bone-liver-kidney type; and more preferably also comprise expression of one or more additional bone markers such as osteocalcin (OCN), procollagen type 1 amino-terminal propeptide (P1NP), osteonectin (ON), osteopontin (OP) and/or bone sialoprotein (BSP), and/or one or more additional bone matrix proteins such as decorin and/or osteoprotegerin (OPG);
c) the cells substantially do not express CD45 (e.g., less than about 10%, preferably less than about 5%, more preferably less than about 2% of the cells may express CD45);
d) the cells show evidence of ability to mineralize the external surroundings, or synthesize calcium-containing extracellular matrix (e.g., when exposed to osteogenic medium; see Jaiswal et al. J Cell Biochem, 1997, vol. 64, 295-312). Calcium accumulation inside cells and deposition into matrix proteins can be conventionally measured for example by culturing in ⁴⁵Ca²⁺, washing and re-culturing, and then determining any radioactivity present inside the cell or deposited into the extracellular matrix (U.S. Pat. No. 5,972,703), or using an Alizarin red-based mineralization assay (see, e.g., Gregory et al. Analytical Biochemistry, 2004, vol. 329, 77-84);
e) the cells substantially do not differentiate towards neither of cells of adipocytic lineage (e.g., adipocytes) or chondrocytic lineage (e.g., chondrocytes). The absence of differentiation towards such cell lineages may be tested using standard differentiation inducing conditions established in the art (e.g., see Pittenger et al. Science, 1999, vol. 284, 143-7), and assaying methods (e.g., when induced, adipocytes typically stain with oil red O showing lipid accumulation; chondrocytes typically stain with alcian blue or safranin O). Substantially lacking propensity towards adipogenic and/or chondrogenic differentiation may typically mean that less than 20%, or less than 10%, or less than 5%, or less than 1% of the tested cells would show signs of adipogenic or chondrogenic differentiation when applied to the respective test.

The cells may further comprise expression of one or more cell recruitment factors such as VEGF. The cells may further comprise expression of IL6.

Cells classified as constituting or belonging to osteocytic (bone) lineage, chondrocytic (cartilage) lineage, adipocytic (fat) lineage, myocytic (muscle) lineage, tendonocytic (tendon) lineage, fibroblastic (connective tissue) lineage, or stromogenic (stroma) lineage, are well-known to those skilled in the art. They encompass cells that have the respective phenotypes, and that can contribute to, or are capable of developing to cells which can contribute to, the formation of the respective tissue types. By means of further guidance and example, cells of osteocytic lineage include osteoprogenitors, such as early and late osteoprogenitors, pre-osteoblasts, osteoblasts and osteocytes; cells of chondrocytic lineage include chondroblasts and chondrocytes, the latter also encompassing hypertrophic chondrocytes; cells of adipocytic lineage include adipoblasts (or preadipocytes) and adipocytes; cells of myocytic lineage include satellite cells, myoblasts, and myocytes (cells of any type of muscle tissue, i.e., cardiac, skeletal, and smooth muscle tissue, are envisaged, more preferably cells of skeletal muscle tissue); cells of tendonocytic lineage include tenoblasts and tenocytes; cells of fibroblastic lineage include fibrocytes and fibroblasts; cells of stromogenic lineage include stromal cells, such as in particular bone marrow stromal cells.

Wherein a cell is said to be positive for (or to express or comprise expression of) a particular marker, this means that a skilled person will conclude the presence or evidence of a distinct signal, e.g., antibody-detectable or detection by reverse transcription polymerase chain reaction, for that marker when carrying out the appropriate measurement, compared to suitable controls. Where the method allows for quantitative assessment of the marker, positive cells may on average generate a signal that is significantly different from the control, e.g., but without limitation, at least 1.5-fold higher than such signal generated by control cells, e.g., at least 2-fold, at least 4-fold, at least 10-fold, at least 20-fold, at least 30-fold, at least 40-fold, at least 50-fold higher or even higher.

The expression of the above cell-specific markers can be detected using any suitable immunological technique known in the art, such as immuno-cytochemistry or affinity adsorption, Western blot analysis, FACS, ELISA, etc., or by any suitable biochemical assay of enzyme activity (e.g., for ALP), or by any suitable technique of measuring the quantity of the marker mRNA, e.g., Northern blot, semi-quantitative or quantitative RT-PCR, etc. Sequence data for markers listed in this disclosure are known and can be obtained from public databases such as GenBank (http://www.ncbi.nlm.nih.gov/).

As mentioned, the invention also contemplates cell populations comprising osteoprogenitors or osteoblastic cells. An exemplary cell population may comprise at least 10%, preferably at least 30%, more preferably at least 50%, e.g., at least 60%, yet more preferably at least 70%, e.g., at least 80%, and even more preferably at least 90%, e.g., at least 95% of osteoprogenitors and/or osteoblastic cells as taught herein. For example, the cell population may comprise less than 50%, preferably less than 40%, even more preferably less than 30%, yet more preferably less than 20% and still more preferably less than 10%, e.g., less than 7%, less than 5% or less than 2% of cell types other than the osteoprogenitors and osteoblastic cells as defined herein.

The term “stem cell” refers generally to an unspecialized or relatively less specialized and proliferation-competent cell, which is capable of self-renewal, i.e., can proliferate without differentiation, and which or the progeny of which can give rise to at least one relatively more specialized cell type. The term encompasses stem cells capable of substantially unlimited self-renewal, i.e., wherein the progeny of a stem cell or at least part thereof substantially retains the unspecialized or relatively less specialized phenotype, the differentiation potential, and the proliferation capacity of the mother stem cell, as well as stem cells which display limited self-renewal, i.e., wherein the capacity of the progeny or part thereof for further proliferation and/or differentiation is demonstrably reduced compared to the mother cell. By means of example and not limitation, a stem cell may give rise to descendants that can differentiate along one or more lineages to produce increasingly relatively more specialized cells, wherein such descendants and/or increasingly relatively more specialized cells may themselves be stem cells as defined herein, or even to produce terminally differentiated cells, i.e., fully specialized cells, which may be post-mitotic.

The term “adult stem cell” as used herein refers to a stem cell present in or obtained from (such as isolated from) an organism at the foetal stage or after birth, such as for example after achieving adulthood.

Preferable stem cells according to the invention have the potential of generating cells of at least the osteogenic (bone) lineage, such as, e.g., osteogenic cells and/or osteoprogenitors and/or pre-osteoblasts and/or osteoblasts and/or osteocytes, etc.

Preferably, at least some stem cells according to the invention may also have the potential to generate further cells comprised in the cell populations resulting from the present methods, such as, e.g., cells of endothelial lineage, for example endothelial progenitor cells and/or endothelial cells.

The term “mesenchymal stem cell” or “MSC”, as used herein, refers to an adult, mesoderm-derived stem cell that is capable of generating cells of mesenchymal lineages, typically of two or more mesenchymal lineages, e.g., osteocytic (bone), chondrocytic (cartilage), myocytic (muscle), tendonocytic (tendon), fibroblastic (connective tissue), adipocytic (fat) and stromogenic (marrow stroma) lineage. MSC may be isolated from, e.g., bone marrow, trabecular bone, blood, umbilical cord, placenta, foetal yolk sac, skin (dermis), specifically foetal and adolescent skin, periosteum and adipose tissue. Human MSC, their isolation, in vitro expansion, and differentiation, have been described in, e.g., U.S. Pat. No. 5,486,359; U.S. Pat. No. 5,811,094; U.S. Pat. No. 5,736,396; U.S. Pat. No. 5,837,539; or U.S. Pat. No. 5,827,740. Any MSC described in the art and isolated by any method described in the art may be suitable in the present methods, provided such MSC are capable of generating cells of at least the osteocytic (bone) lineage.

The term MSC also encompasses the progeny of MSC, e.g., progeny obtained by in vitro or ex vivo propagation of MSC obtained from a biological sample of an animal or human subject.

Potentially, but without limitation, at least some MSC might also be able to generate further cells comprised in the cell populations resulting from the present methods.

As shown in the examples, the method of certain aspects of the invention may entail selecting those bone marrow stem cells (BMSC) which under the specified culture conditions adhere to a substrate surface. It is known in the art that MSC can be isolated from bone marrow (or other sources) by selecting those (mononuclear) cells which can adhere to a substrate surface, e.g., plastic surface. Hence, preferably, MSC as used herein may be isolated from bone marrow. A sample of bone marrow (BMSC) may be obtained, e.g., from iliac crest, femora, tibiae, spine, rib or other medullar spaces of a subject.

The term “isolating” with reference to a particular component denotes separating that component from at least one other component of a composition from which the former component is being isolated. The term “isolated” as used herein in relation to any cell type or cell population also implies that such cell population does not form part of an animal or human body.

MSC may be comprised in a biological sample, e.g., in a sample comprising BMSC, or may be at least partly isolated there from as known in the art. Moreover, MSC may be at least partly isolated from bone marrow or from any suitable sources comprising MSC other than bone marrow, e.g., blood, umbilical cord, placenta, foetal yolk sac, skin (dermis), specifically foetal and adolescent skin, periosteum and adipose tissue.

The term “in vitro” generally denotes outside, or external to, animal or human body. The term “ex vivo” typically refers to tissues or cells removed from an animal or human body and maintained or propagated outside the body, e.g., in a culture vessel. The term “in vitro” as used herein should be understood to include “ex vivo”. The term “in vivo” generally denotes inside, on, or internal to, animal or human body.

In an embodiment, MSC or other cell types as envisaged herein may be obtained from a biological sample of a subject.

The term “subject” or “patient” are used interchangeably and refer to animals, preferably warm-blooded animals, more preferably vertebrates, and even more preferably mammals specifically including humans and non-human mammals, that have been the object of treatment, observation or experiment. The term “mammal” includes any animal classified as such, including, but not limited to, humans, domestic and farm animals, zoo animals, sport animals, pet animals, companion animals and experimental animals, such as, for example, mice, rats, hamsters, rabbits, dogs, cats, guinea pigs, cattle, cows, sheep, horses, pigs and primates, e.g., monkeys and apes. Particularly preferred are human subjects, including both genders and all age categories thereof.

Hence, also provided is a method for differentiating adult human MSC in vitro or ex vivo into osteoprogenitors or osteoblastic cells or a cell population comprising osteoprogenitors and/or osteoblastic cells, the method comprising the step of culturing said MSC in a medium comprising human plasma or serum, GDF-8 and FGF-2.

Non-human animal subjects may also include prenatal forms of animals, such as, e.g., embryos or foetuses. Human subjects may also include foetuses, but by preference not embryos.

The term “biological sample” or “sample” as used herein generally refers to a sample obtained from a biological source, e.g., from an organism, organ, tissue or cell culture, etc. A biological sample of an animal or human subject refers to a sample removed from an animal or human subject and comprising cells thereof. The biological sample of an animal or human subject may comprise one or more tissue types and cells of one or more tissue types. Methods of obtaining biological samples of an animal or human subject are well known in the art, e.g., tissue biopsy or drawing blood.

A useful biological sample of a subject comprises MSC thereof or other cell types as envisaged herein of the subject. A sample comprising MSC may be typically obtained from bone marrow, e.g., from iliac crest, femora, tibiae, spine, rib or other medullar spaces of a subject. Another useful biological sample comprising MSC may be derived, e.g., from blood, umbilical cord, placenta, foetal yolk sac, skin (dermis), specifically foetal and adolescent skin, periosteum, trabecular bone or adipose tissue of a subject. Other cell types as discussed herein may be isolated using available protocols from the corresponding tissues in which they reside, e.g., osteocytic lineage cells from bone tissue, chondrocytic lineage cells from cartilage tissue, adipocytic lineage cells from fat tissue, myocytic lineage cells from smooth, cardiac or skeletal (preferably skeletal) muscle tissue, tendonocytic lineage cells from tendon tissue, fibroblastic lineage cells from connective tissue, or stromogenic lineage cells from stromal tissue, such as bone marrow stroma. Alternatively, such cell types may be differentiated from MSC using protocols known per se.

In an embodiment, MSC may be obtained from a healthy subject, which may help to ensure the functionality of the osteoprogenitors or osteoblastic cells or the cell population comprising osteoprogenitors and/or osteoblastic cells or other cell types as envisaged herein, which are differentiated from said MSC. In a further embodiment, MSC or other cell types as envisaged herein may be obtained from a healthy subject, which may help to ensure the functionality of such cells.

In another embodiment, MSC may be obtained from a subject who is at risk for or has a musculoskeletal disease such as for instance a bone disease, and who can thus particularly benefit from administration of the osteoprogenitors or osteoblastic cells or the cell population comprising osteoprogenitors and/or osteoblastic cells, differentiated from said MSC according to the methods of the present invention.

In a further embodiment, MSC or other cell types as envisaged herein may be obtained from a subject who is at risk for or has a disease detrimentally affecting a tissue which can benefit from administration of the MSC or of the one or more other cell types as envisaged herein.

The term “musculoskeletal disease”, as used herein refers to any type of bone disease, muscle disease, osteoarticular disease, or chondrodystrophy, the treatment of which may benefit from the administration of osteogenic lineage cells, e.g., osteoprogenitors or osteoblastic cells or a cell population comprising osteoprogenitors and/or osteoblastic cells to a subject having the disease. In particular, such disease may be characterized, e.g., by decreased bone formation or excessive bone resorption, by decreased number, viability or function of osteoblasts or osteocytes present in the bone, decreased bone mass in a subject, thinning of bone, compromised bone strength or elasticity, etc.

Non-limiting examples of musculoskeletal diseases which can benefit from administration of osteoprogenitors or osteoblastic cells or a cell population comprising osteoprogenitors and/or osteoblastic cells as taught herein may include local or systemic disorders, such as, any type of osteoporosis or osteopenia, e.g., primary, postmenopausal, senile, corticoid-induced, bisphosphonates-induced, and radiotherapy-induced; any secondary, mono- or multisite osteonecrosis; any type of fracture, e.g., non-union, mal-union, delayed union fractures or compression, conditions requiring bone fusion (e.g., spinal fusions and rebuilding), maxillo-facial fractures, congenital bone defect, bone reconstruction, e.g., after traumatic injury or cancer surgery, and cranio-facial bone reconstruction; traumatic arthritis, focal cartilage and/or joint defect, focal degenerative arthritis; osteoarthritis, degenerative arthritis, gonarthrosis, and coxarthrosis; osteogenesis imperfecta; osteolytic bone cancer; Paget's Disease, endocrinological disorders, hypophosphatemia, hypocalcemia, renal osteodystrophy, osteomalacia, adynamic bone disease, hyperparathyroidism, primary hyperparathyroidism, secondary hyperparathyroidism; periodontal disease; Gorham-Stout disease and McCune-Albright syndrome; rheumatoid arthritis; spondyloarthropathies, including ankylosing spondylitis, psoriatic arthritis, enteropathic arthropathy, and undifferentiated spondyloarthritis and reactive arthritis; systemic lupus erythematosus and related syndromes; scleroderma and related disorders; Sjogren's Syndrome; systemic vasculitis, including Giant cell arteritis (Horton's disease), Takayasu's arteritis, polymyalgia rheumatica, ANCA-associated vasculitis (such as Wegener's granulomatosis, microscopic polyangiitis, and Churg-Strauss Syndrome), Behcet's Syndrome, and other polyarteritis and related disorders (such as polyarteritis nodosa, Cogan's Syndrome, and Buerger's disease); arthritis accompanying other systemic inflammatory diseases, including amyloidosis and sarcoidosis; crystal arthropathies, including gout, calcium pyrophosphate dihydrate disease, disorders or syndromes associated with articular deposition of calcium phosphate or calcium oxalate crystals; chondrocalcinosis and neuropathic arthropathy; Felty's Syndrome and Reiter's Syndrome; Lyme disease and rheumatic fever.

The methods and uses applying the principles of the present invention may concern culturing (e.g., maintaining, propagating and/or differentiating) cells or cell populations in the presence of cell or tissue culture media as known per se, such as for example using liquid or semi-solid (e.g., gelatinous), and preferably liquid cell or tissue culture media. Such culture media can desirably sustain the maintenance (e.g., survival, genotypic, phenotypic and/or functional stability) and propagation of the cells or cell populations.

In particular, methods embodying the principles of the present invention may comprise the step of culturing MSC in a medium comprising plasma or serum and GDF-8 and FGF-2. Hence, generally speaking, cells may be cultured in a medium comprising one or more agents, such as growth factors and plasma or serum, by means of their inclusion in the medium.

A skilled person appreciates that plasma and serum are complex biological compositions, which may comprise one or more growth factors, cytokines or hormones. Hence, it is intended that the recited growth factors, in particular GDF-8 and FGF-2, are provided in addition to, i.e., exogenously to or in supplement to, the plasma or serum.

Also provided are methods for differentiating adult mesenchymal stem cells (MSC) in vitro or ex vivo into osteoprogenitors or osteoblastic cells or a cell population comprising osteoprogenitors and/or osteoblastic cells, the method comprising the step of culturing said MSC in a medium consisting essentially of or consisting of basal medium, plasma or serum, GDF-8 and FGF-2.

Hence, in an embodiment, GDF-8 and FGF-2 may be the sole growth factors added to the medium.

In a further embodiment, MSC may be cultured, in addition to GDF-8 and FGF-2, with one or more additional, exogenously added growth factors other than GDF-8 and FGF-2.

In a preferred embodiment, any one or more or all growth factors used in the present method (general reference to a growth factor as used herein particularly encompasses the GDF-8 and FGF-2 growth factors, as well as the optional one or more further growth factors) is human growth factor. As used herein, the term “human growth factor” refers to a growth factor substantially the same as a naturally occurring human growth factor. For example, where the growth factor is a proteinaceous entity, the constituent peptide(s) or polypeptide(s) thereof may have primary amino acid sequence identical to a naturally occurring human growth factor. The use of human growth factors is preferred as such growth factors are expected to elicit a desirable effect on human cellular function.

The term “naturally occurring” is used to describe an object or entity that can be found in nature as distinct from being artificially produced by man. For example, a polypeptide sequence present in an organism, which can be isolated from a source in nature and which has not been intentionally modified by man in the laboratory, is naturally occurring. When referring to a particular entity, e.g., to a polypeptide or protein, the term encompasses all forms and variants thereof which occur in nature, e.g., due to a normal variation between species and individuals. For example, when referring to a proteinaceous growth factor, the term “naturally occurring” encompasses growth factors having differences in the primary sequence of their constituent peptide(s) or polypeptide(s) due to genetic divergence between species and normal allelic variation between individuals.

FGF-2 or fibroblast growth factor 2 is also commonly known as basic fibroblast growth factor, FGFb, bFGF, prostatropin, or heparin-binding growth factor 2 precursor (HBGF-2). Exemplary human FGF-2 includes, without limitation, FGF-2 having primary amino acid sequence as annotated under Uniprot/Swissprot accession number P09038 (http://www.uniprot.org/uniprot/). A skilled person can appreciate that said sequence is of a precursor FGF-2 and may include parts which are processed away from mature FGF-2. Exemplary human FGF-2 protein sequence may be as annotated under NCBI Genbank (http://www.ncbi.nlm.nih.gov/) accession number NP_{—}001997.5. Exemplary human FGF-2 has been also described inter alia by Abraham et al. 1986 (EMBO J 5: 2523-8) and Kurokawa et al. 1987 (FEBS Lett 213: 189-94).

GDF-8 or growth and differentiation factor 8 is also commonly known as myostatin (MSTN). Exemplary human GDF-8 includes, without limitation, GDF-8 having primary amino acid sequence as annotated under Uniprot/Swissprot accession number O14793. Exemplary human GDF-8 protein precursor sequence may be as annotated under NCBI Genbank (http://www.ncbi.nlm.nih.gov/) accession number NP_{—}005250.1. Exemplary murine GDF-8 has been also described inter alia by McPherron et al. 1997 (Nature 387 (6628): 83-90).

GDF-8 and FGF-2 are comprised in a medium as defined herein at concentrations sufficient to induce differentiation of MSC into osteoprogenitors or osteoblastic cells or a cell population comprising osteoprogenitors and/or osteoblastic cells. Typically, FGF-2 can be included in the medium at a concentration of between 0.1 and 100 ng/ml, preferably between 0.5 and 20 ng/ml, e.g., at about 19, 18, 17, 16, 15, 14, 13, 12, 11, 10, 9, 8, 7 or 6 ng/ml, or at about 5 ng/ml or less, e.g., at about 4, 3, 2, 1 or 0.5 ng/ml. Typically, GDF-8 can be included in the medium at a concentration of between 0.1 and 1000 ng/ml, for example between 1 and 500 ng/ml, e.g., at about 450, 400, 350, 300, 250 or 200 ng/ml, or at about 150 ng/ml or less, e.g., at about 100, 75, 50, 25 or 10 ng/ml, or preferably at about 5 ng/ml or less, e.g., at about 4, 3, 2, 1, 0.75 0.5 or 0.25 ng/ml. Said values are intended to refer to concentrations of the respective growth factors as exogenously supplemented to the media.

The reference herein to any protein, polypeptide or peptide such as any growth factor including GDF-8 and FGF-2 may also encompass fragments thereof. The term “fragment” of a protein, polypeptide or peptide generally refers to N-terminally and/or C-terminally deleted or truncated forms of said protein, polypeptide or peptide. Without limitation, a fragment of a nucleic acid, protein, polypeptide or peptide may represent at least about 5%, or at least about 10%, e.g., ≧20%, ≧30% or ≧40%, such as preferably ≧50%, e.g., ≧60%, ≧70% or ≧80%, or more preferably ≧90% or ≧95% of the nucleotide sequence of said nucleic acid or of the amino acid sequence of said protein, polypeptide or peptide.

The reference herein to any protein, polypeptide or peptide such as any growth factor including GDF-8 and FGF-2 may also encompass variants thereof. The term “variant” of a nucleic acid, protein, polypeptide or peptide refers to nucleic acid, proteins, polypeptides or peptides the sequence (i.e., nucleotide sequence or amino acid sequence, respectively) of which is substantially identical (i.e., largely but not wholly identical) to the sequence of said recited nucleic acid, protein or polypeptide, e.g., at least about 80% identical or at least about 85% identical, e.g., preferably at least about 90% identical, e.g., at least 91% identical, 92% identical, more preferably at least about 93% identical, e.g., at least 94% identical, even more preferably at least about 95% identical, e.g., at least 96% identical, yet more preferably at least about 97% identical, e.g., at least 98% identical, and most preferably at least 99% identical. Preferably, a variant may display such degrees of identity to a recited nucleic acid, protein, polypeptide or peptide when the whole sequence of the recited nucleic acid, protein, polypeptide or peptide is queried in the sequence alignment (i.e., overall sequence identity). Also included among fragments and variants of a nucleic acid, protein, polypeptide or peptide are fusion products of said nucleic acid, protein, polypeptide or peptide with another, usually unrelated, nucleic acid, protein, polypeptide or peptide.

Sequence identity may be determined using suitable algorithms for performing sequence alignments and determination of sequence identity as know per se. Exemplary but non-limiting algorithms include those based on the Basic Local Alignment Search Tool (BLAST) originally described by Altschul et al. 1990 (J Mol Biol 215: 403-10), such as the “Blast 2 sequences” algorithm described by Tatusova and Madden 1999 (FEMS Microbiol Lett 174: 247-250), for example using the published default settings or other suitable settings (such as, e.g., for the BLASTN algorithm: cost to open a gap=5, cost to extend a gap=2, penalty for a mismatch=−2, reward for a match=1, gap x_dropoff=50, expectation value=10.0, word size=28; or for the BLASTP algorithm: matrix=Blosum62, cost to open a gap=11, cost to extend a gap=1, expectation value=10.0, word size=3).

A variant of a protein, polypeptide or peptide may be a homologue (e.g., orthologue or paralogue) of said protein, polypeptide or peptide. As used herein, the term “homology” generally denotes structural similarity between two macromolecules, particularly between two proteins or polypeptides, from same or different taxons, wherein said similarity is due to shared ancestry.

Where the present specification refers to or encompasses fragments and/or variants of proteins, polypeptides or peptides, this preferably denotes variants and/or fragments which are “functional”, i.e., which at least partly retain the biological activity or intended functionality of the respective proteins, polypeptides or peptides. By means of an example and not limitation, a functional fragment and/or variant of GDF-8 or FGF-2 shall at least partly retain the biological activity of GDF-8 or FGF-2, respectively. For example, it may retain one or more aspects of the biological activity of GDF-8 or FGF-2, such as, e.g., ability to bind to one or more cognate receptors, to participate in one or more cellular pathways, etc. Preferably, a functional fragment and/or variant may retain at least about 20%, e.g., at least 30%, or at least about 40%, or at least about 50%, e.g., at least 60%, more preferably at least about 70%, e.g., at least 80%, yet more preferably at least about 85%, still more preferably at least about 90%, and most preferably at least about 95% or even about 100% or higher of the intended biological activity or functionality compared to the corresponding protein, polypeptide or peptide. Particularly, a functional fragment or variant would retain, to at least a certain degree, the ability to stimulate osteogenic differentiation of MSC cells in the present methods or uses.

Where a protein, polypeptide or peptide such as a growth factor exerts its effects by binding to its cognate receptor, a functional fragment and/or variant of the protein, polypeptide or peptide may retain at least about 20%, e.g., at least 30%, or at least about 40%, or at least about 50%, e.g., at least 60%, more preferably at least about 70%, e.g., at least 80%, yet more preferably at least about 85%, still more preferably at least about 90%, and most preferably at least about 95% or even about 100% or higher of the affinity and/or specificity of the respective protein, polypeptide or peptide for binding to that receptor. The above parameters of the binding may be readily determined by a skilled person using in vitro or cellular assays which are known per se.

Where the activity of a given protein, polypeptide or peptide such as a given growth factor can be readily measured in an established assay, e.g., an in vitro or cellular assay (such as, for example, measurement of mitogenic activity in cell culture), a functional fragment and/or variant of the protein, polypeptide or peptide may display activity in such assays, which is at least about 20%, e.g., at least 30%, or at least about 40%, or at least about 50%, e.g., at least 60%, more preferably at least about 70%, e.g., at least 80%, yet more preferably at least about 85%, still more preferably at least about 90%, and most preferably at least about 95% or even about 100% or higher of the activity of the respective protein, polypeptide or peptide.

Reference to the “activity” of a protein, polypeptide or peptide such as a growth factor may generally encompass any one or more aspects of the biological activity of the protein, polypeptide or peptide, such as without limitation any one or more aspects of its biochemical activity, enzymatic activity, signalling activity, interaction activity, ligand activity, and/or structural activity, e.g., within a cell, tissue, organ or an organism. By means of an example and not limitation, reference to the activity of GDF-8 or FGF-2 may particularly denote their activity as a ligand, i.e., their ability to bind to one or more cognate receptors, and/or their activity as a signalling molecule, i.e., their ability to participate in one or more cellular signalling pathways, etc.

The reference herein to any protein, polypeptide or peptide such as any growth factor including GDF-8 and FGF-2 may also encompass derivatives thereof. The term “derivative” of a protein, polypeptide or peptide generally refers to a protein, polypeptide or peptide derivatised by chemical alteration of one or more amino acid residues and/or addition of one or more moieties at one or more amino acid residues, e.g., by glycosylation, phosphorylation, acylation, acetylation, sulphation, lipidation, alkylation, etc. Typically, less than 50%, e.g., less than 40%, preferably less than 30%, e.g., less than 20%, more preferably less than 15%, e.g., less than 10% or less than 5%, e.g., less than 4%, 3%, 2% or 1% of amino acids in the protein, polypeptide or peptide may be derivatised. A proteinaceous derivative may be comprised of one or more protein(s), polypeptide(s) or peptide(s), at least one of which may be derivatised on at least one amino acid residue. Where the present specification refers to or encompasses derivatives of proteins, polypeptides or peptides, this preferably denotes derivatives which are functional.

In a preferred embodiment, the growth factor may be recombinant, i.e., produced by a host organism through the expression of a recombinant nucleic acid molecule, which has been introduced into the host organism (e.g., bacteria such as without limitation E. coli, S. tymphimurium, Serratia marcescens, Bacillus subtilis; yeast such as for example S. cerevisiae and Pichia pastoris; cultured plant cells such as inter alia Arabidopsis thaliana and Nicotiana tobaccum cells; animal cells such as mammalian and insect cells; or multi-cellular organisms such as plants or animals) or an ancestor thereof, and which comprises a sequence encoding the growth factor. The use of recombinantly expressed growth factors lowers the risk of transmission of pathogenic agents.

The term “plasma” is as conventionally defined. Plasma is usually obtained from a sample of whole blood, provided or contacted with an anticoagulant, (e.g., heparin, citrate, oxalate or EDTA). Subsequently, cellular components of the blood sample are separated from the liquid component (plasma) by an appropriate technique, typically by centrifugation. The term “plasma” therefore refers to a composition which does not form part of a human or animal body.

The term “serum” is as conventionally defined. Serum can be usually obtained from a sample of whole blood by first allowing clotting to take place in the sample and subsequently separating the so formed clot and cellular components of the blood sample from the liquid component (serum) by an appropriate technique, typically by centrifugation. Clotting can be facilitated by an inert catalyst, e.g., glass beads or powder. Alternatively, serum can be obtained from plasma by removing the anticoagulant and fibrin. The term “serum” hence refers to a composition which does not form part of a human or animal body.

The isolated plasma or serum can be used directly in the present method. They can also be appropriately stored for later use (e.g., for shorter time periods, e.g., up to about 1-2 weeks, at a temperature above the respective freezing points of plasma or serum, but below ambient temperature, this temperature will usually be about 4° C. to 5° C.; or for longer times by freeze storage, usually at between about −70° C. and about −80° C.).

The isolated plasma or serum can be heat inactivated as known in the art, particularly to remove the complement. Where the present method employs plasma or serum autologous to the cells cultured in the presence thereof, it may be unnecessary to heat inactivate the plasma or serum. Where the plasma or serum is at least partly allogeneic to the cultured cells, it may be advantageous to heat inactivate the plasma or serum.

Optionally, the plasma or serum may also be sterilized prior to storage or use, using conventional microbiological filters, preferably with pore size of 0.2 μm or smaller.

The method of the present invention may employ plasma or serum which is autologous to MSC or other cell types contacted therewith. The term “autologous” with reference to plasma or serum denotes that the plasma or serum is obtained from the same subject as are the MSC or other cell types to be contacted with the plasma or serum. The method of the present invention may further employ plasma or serum which is “homologous” or “allogeneic” to MSC or other cell types contacted therewith, i.e., obtained from one or more (pooled) subjects other than the subject from which the MSC or other cell types are obtained. The method of the present invention may also employ a mixture of autologous and homologous (allogeneic) plasma or sera as defined above.

Also disclosed are methods for differentiating adult MSC in vitro or ex vivo into osteoprogenitors or osteoblastic cells or a cell population comprising osteoprogenitors and/or osteoblastic cells, the method comprising the step of culturing said MSC in a medium comprising plasma or serum and GDF-8. As illustrated in the example section, a method comprising the step of culturing MSC in a medium comprising plasma or serum and GDF-8 at least partly achieves the advantageous effects as obtained with the present methods comprising the step of culturing said MSC in a medium comprising plasma or serum and GDF-8 and FGF-2.

In one embodiment, the methods of the present invention comprise the steps of: (a) allowing cells recovered from a biological sample of a subject and comprising MSC to adhere to a substrate surface; and (b) culturing adherent cells in a medium comprising plasma or serum, GDF-8 and FGF-2.

Any one of the methods of the present invention may optionally comprise the step of isolating mono-nucleated cells from the cells recovered from a biological sample of a subject and comprising MSC prior to allowing the so-isolated mono-nucleated cells to adhere to the substrate surface. Isolation of mono-nucleated cells may be performed using conventional methods such as, e.g., density gradient centrifugation.

Culturing of cells, such as in particular adherent cells, is performed in the presence of a medium, commonly a liquid cell culture medium. Typically, the medium will comprise a basal medium formulation as known in the art. Many basal media formulations (available, e.g., from the American Type Culture Collection, ATCC; or from Invitrogen, Carlsbad, Calif.) can be used to culture the cells herein, including but not limited to Eagle's Minimum Essential Medium (MEM), Dulbecco's Modified Eagle's Medium (DMEM), alpha modified Minimum Essential Medium (alpha-MEM), Basal Medium Essential (BME), BGJb, F-12 Nutrient Mixture (Ham), Iscove's Modified Dulbecco's Medium (IMDM), available from Invitrogen or Cambrex (New Jersey), and modifications and/or combinations thereof. Compositions of the above basal media are generally known in the art and it is within the skill of one in the art to modify or modulate concentrations of media and/or media supplements as necessary for the cells cultured.

The cells can be allowed to adhere to a substrate surface as intended herein in the presence of said medium.

Such basal media formulations contain ingredients necessary for mammalian cell development, which are known per se. By means of illustration and not limitation, these ingredients may include inorganic salts (in particular salts containing Na, K, Mg, Ca, Cl, P and possibly Cu, Fe, Se and Zn), physiological buffers (e.g., HEPES, bicarbonate), nucleotides, nucleosides and/or nucleic acid bases, ribose, deoxyribose, amino acids, vitamins, antioxidants (e.g., glutathione) and sources of carbon (e.g. glucose, sodium pyruvate, sodium acetate), etc.

For use in culture, basal media can be supplied with one or more further components. For example, additional supplements can be used to supply the cells with the necessary trace elements and substances for optimal growth and expansion. Furthermore, antioxidant supplements may be added, e.g., β-mercaptoethanol. While many basal media already contain amino acids, some amino acids may be supplemented later, e.g., L-glutamine, which is known to be less stable when in solution. A medium may be further supplied with antibiotic and/or antimycotic compounds, such as, typically, mixtures of penicillin and streptomycin, and/or other compounds, exemplified but not limited to, amphotericin, ampicillin, gentamicin, bleomycin, hygromycin, kanamycin, mitomycin, mycophenolic acid, nalidixic acid, neomycin, nystatin, paromomycin, polymyxin, puromycin, rifampicin, spectinomycin, tetracycline, tylosin, and zeocin.

Lipids and lipid carriers can also be used to supplement cell culture media. Such lipids and carriers can include, but are not limited to cyclodextrin, cholesterol, linoleic acid conjugated to albumin, linoleic acid and oleic acid conjugated to albumin, unconjugated linoleic acid, linoleic-oleic-arachidonic acid conjugated to albumin, oleic acid unconjugated and conjugated to albumin, among others. Albumin can similarly be used in fatty-acid free formulations.

Plasma or serum may also be comprised in said media at a proportion (volume of plasma or serum/volume of medium) between about 0.5% and about 30%, preferably between about 1% and about 15%. The present methods may perform satisfactorily with relatively low amounts of plasma or serum, e.g., about 5 or 10 volume % or below, e.g. about 1, about 2, about 3 or about 4 volume %, advantageously reducing cost or allowing to decrease the volume of plasma or serum that needs to be obtained in order to culture the MSC.

In one embodiment of the present invention, the cells (esp. the cells of (a)) may be plated for adherence at between 5×10² and 5×10⁷ cells/cm², preferably between 5×10³ and 5×10⁵ cells/cm².

The culture vessel may provide for a plastic surface to enable cell adherence. The surface may be a glass surface or may be coated with an appropriate material conducive to adherence and growth of cells, e.g., Matrigel(R), laminin or collagen.

The cells can be cultured in the medium as defined in (b) for a period of between about 10 and about 18 days. For instance, the cells can be cultured in step (b) or in steps (a) and (b) taken together for a period of between about 10 and about 16 days, usually between about 12-14 days. Otherwise, the cells may be cultured in step (b) or in step (a) and (b) taken together until their confluence reaches about 60% or more, or about 80% or more, or about 90% or more, or even up to 100%.

In an embodiment, following step (b) the method may comprise collecting the so-obtained cells or cell population.

In another embodiment, following step (b) the acquired cells may be passaged one or more times, such as one, two, three, four, or more times and preferably, the cells may be passaged one, two or three times, more preferably, one or two times, even more preferably, the cells may be passaged once. The passage number refers to the number of times that a cell population has been removed from a culture vessel and undergone a subculture, i.e., a passage. For example, passaging may usually include detachment of cells using a bivalent ion chelator (e.g., EDTA or EGTA) and/or trypsin or suitable protease; re-suspending the detached cells; and re-plating the cells in same or a new culture vessel at a desired cell density.

In a preferred embodiment, subsequent to step (b), the method thus further includes step (c) passaging, in particular once, and further culturing the cells or cell population from step (b) in the medium as defined in (b). Following such step (c) the cells or cell populations may be collected.

In the passaging step (c), the cells are preferably plated for further culturing at between 5×10¹ and 5×10⁶ cells/cm², preferably between 5×10² and 5×10⁴ cells/cm², more typically at about 5×10³ cells/cm².

Typically, the cells are further cultured in step (c) for a period of between about 3 and about 18 days. This period provides for satisfactory expansion of the cells.

In a further embodiment, the methods of the present invention comprise the steps of: (a) allowing cells recovered from a biological sample of a subject and comprising MSC to adhere to a substrate surface; (b′) culturing adherent cells in a medium comprising plasma or serum and FGF-2; and (b″) further culturing adherent cells in a medium comprising plasma or serum, GDF-8 and FGF-2.

The cells may be cultured in the medium as defined in (b′) or in steps (a) and (b′) taken together for a period of between about 10 and about 24 days. For instance, the cells may be cultured in step (b′) or in steps (a) and (b′) taken together for a period of between about 10 and about 22 days, usually between about 14-21 days. Otherwise, the cells may be cultured in step (b′) or in step (a) and (b′) taken together until their confluence reaches about 60% or more, or about 80% or more, or about 90% or more, or even up to 100%.

The cells may be cultured in the medium as defined in (b″) for a period of between about 1 day and about 10 days. For instance, the cells may be cultured in step (b″) for a period of between about 2 days and about 8 days, usually between about 4-6 days. Otherwise, the cells may be cultured in step (b″) until their confluence reaches about 60% or more, or about 80% or more, or about 90% or more, or even up to 100%.

In another embodiment, the method of the present invention further includes between step (b′) and step (b″) the step (c′) of passaging the cells and allowing cells to adhere to a substrate surface. In step (c′), the cells may be passaged one or more times, such as one, two or three times, preferably, the cells may be passaged one or two times.

In the passaging step (c′), the cells are preferably plated for further culturing at between 5×10¹ and 5×10⁶ cells/cm², preferably between 5×10² and 5×10⁴ cells/cm², more typically at about 5×10³ cells/cm².

In another embodiment, following step (b″) the method according to the invention may comprise collecting the so-obtained cells or cell population.

The methods of the present invention yield osteoprogenitors or osteoblastic cells or a cell population comprising osteoprogenitors and/or osteoblastic cells, with superior characteristics, such as in particular high expansion rate and low MHC class II cell surface receptor complex expression, which cells and cell populations are suited for prophylactic or therapeutic treatments such as for implantation.

Accordingly, in a further aspect the invention relates to osteoprogenitors or osteoblastic cells or a cell population comprising osteoprogenitors and/or osteoblastic cells, obtainable or directly obtained using the present methods as described above.

Further provided is an isolated cell population comprising osteoprogenitors or osteoblastic cells or a cell population comprising osteoprogenitors and/or osteoblastic cells obtainable or directly obtained using the present methods as described above.

The above defined osteoprogenitors or osteoblastic cells or the cell population comprising osteoprogenitors and/or osteoblastic cells embodying the principles of the invention display superior characteristics, such as in particular high expansion rate and low MHC class II cell surface receptor complex expression, which cells and cell populations are suited for prophylactic or therapeutic transplantation.

Accordingly, the osteoprogenitors or osteoblastic cells or the cell population comprising osteoprogenitors and/or osteoblastic cells as taught herein, may be employed for autologous or allogeneic administration to subjects having a musculoskeletal disease (e.g., as defined elsewhere in this specification). Preferably, human osteoprogenitors or osteoblastic cells or the cell population comprising osteoprogenitors and/or osteoblastic cells, can be administered to human subjects for treating musculoskeletal diseases.

As used herein, a phrase such as “a subject in need of treatment” includes subjects that would benefit from treatment of a given condition, particularly musculoskeletal diseases. Such subjects may include, without limitation, those that have been diagnosed with said condition, those prone to contract or develop said condition and/or those in whom said condition is to be prevented.

The terms “treat” or “treatment” encompass both the therapeutic treatment of an already developed disease or condition, such as the therapy of an already developed musculoskeletal diseases, as well as prophylactic or preventive measures, wherein the aim is to prevent or lessen the chances of incidence of an undesired affliction, such as to prevent occurrence, development and progression of musculoskeletal diseases. Beneficial or desired clinical results may include, without limitation, alleviation of one or more symptoms or one or more biological markers, diminishment of extent of disease, stabilised (i.e., not worsening) state of disease, delay or slowing of disease progression, amelioration or palliation of the disease state, and the like. “Treatment” can also mean prolonging survival as compared to expected survival if not receiving treatment.

The term “prophylactically effective amount” refers to an amount of an active compound or pharmaceutical agent that inhibits or delays in a subject the onset of a disorder as being sought by a researcher, veterinarian, medical doctor or other clinician. The term “therapeutically effective amount” as used herein, refers to an amount of active compound or pharmaceutical agent that elicits the biological or medicinal response in a subject that is being sought by a researcher, veterinarian, medical doctor or other clinician, which may include inter alia alleviation of the symptoms of the disease or condition being treated. Methods are known in the art for determining therapeutically and prophylactically effective doses for the present osteoprogenitors or osteoblastic cells or the cell population comprising osteoprogenitors and/or osteoblastic cells.

In one embodiment of the present invention, the osteoprogenitors or osteoblastic cells or the cell population comprising osteoprogenitors and/or osteoblastic cells, or the other cell types envisaged herein, may be differentiated from MSC of a subject into which the osteoprogenitors or osteoblastic cells or the cell population comprising osteoprogenitors and/or osteoblastic cells, or the other cell types envisaged herein, are to be introduced (i.e., autologous cells). According to another embodiment, which may be available herein inter alia due to the low MHC class II cell surface receptor complex expression of the present cells, the osteoprogenitors or osteoblastic cells or the cell population comprising osteoprogenitors and/or osteoblastic cells, or the other cell types envisaged herein, may be differentiated from MSC of one or more subjects other that the subject into which the osteoprogenitors or osteoblastic cells or the cell population comprising osteoprogenitors and/or osteoblastic cells, or the other cell types envisaged herein, are to be introduced (i.e., allogeneic cells).

According to a further aspect of the present invention, the herein defined osteoprogenitors or osteoblastic cells or the cell population comprising osteoprogenitors and/or osteoblastic cells may be formulated into and administered as pharmaceutical compositions.

According to a further aspect of the present invention, GDF-8 may be formulated into and administered as pharmaceutical compositions. Where cells whose immunogenicity is to be reduced by in vivo administration of GDF-8 are also to be administered to a subject, such cells may be suitably formulated into and administered as pharmaceutical compositions. In certain embodiments, the same pharmaceutical composition may comprise both GDF-8 and the cells, whereas in other embodiments, GDF-8 and the cells may be included in separate pharmaceutical compositions. Further, any material whose risk of rejection is to be reduced by in vivo administration of GDF-8 may be suitably formulated into and administered as pharmaceutical compositions. In certain embodiments, the same pharmaceutical composition may comprise both GDF-8 and the material, whereas in other embodiments, GDF-8 and the material may be included in separate pharmaceutical compositions.

Pharmaceutical compositions will typically comprise the one or more active ingredients (e.g., GDF-8, cells and/or materials) and one or more pharmaceutically acceptable carrier/excipient. For example, pharmaceutical compositions may typically comprise the osteoprogenitors or osteoblastic cells or the cell population comprising osteoprogenitors and/or osteoblastic cells as disclosed herein as the active ingredient, and one or more pharmaceutically acceptable carrier/excipient. As used herein, “carrier” or “excipient” includes any and all solvents, diluents, buffers (such as, e.g., neutral buffered saline or phosphate buffered saline), solubilisers, colloids, dispersion media, vehicles, fillers, chelating agents (such as, e.g., EDTA or glutathione), amino acids (such as, e.g., glycine), proteins, disintegrants, binders, lubricants, wetting agents, emulsifiers, sweeteners, colorants, flavourings, aromatisers, thickeners, agents for achieving a depot effect, coatings, antifungal agents, preservatives, stabilisers, antioxidants, tonicity controlling agents, absorption delaying agents, and the like. The use of such media and agents for pharmaceutical active substances is well known in the art. Such materials should be non-toxic and should not interfere with the activity of the cells.

The precise nature of the carrier or other material will depend on the route of administration. For example, the composition may be in the form of a parenterally acceptable aqueous solution, which is pyrogen-free and has suitable pH, isotonicity and stability. For general principles in medicinal formulation, the reader is referred to the Handbook of Pharmaceutical Excipients 6^{th} Edition 2009, eds. Rowe et al; Remington's Pharmaceutical Sciences, 18th ed., Mack Publishing Co., Easton, Pa. (1990); Cell Therapy: Stem Cell Transplantation, Gene Therapy, and Cellular Immunotherapy, by G. Morstyn & W. Sheridan eds., Cambridge University Press, 1996; and Hematopoietic Stem Cell Therapy, E. D. Ball, J. Lister & P. Law, Churchill Livingstone, 2000.

Such pharmaceutical compositions may contain further components ensuring the viability of the cells therein. For example, the compositions may comprise a suitable buffer system (e.g., phosphate or carbonate buffer system) to achieve desirable pH, more usually near neutral pH, and may comprise sufficient salt to ensure isoosmotic conditions for the cells to prevent osmotic stress. For example, suitable solution for these purposes may be phosphate-buffered saline (PBS), sodium chloride solution, Ringer's Injection or Lactated Ringer's Injection, as known in the art. Further, the composition may comprise a carrier protein, e.g., albumin, which may increase the viability of the cells.

The pharmaceutical compositions according to the present invention may also comprise further components with osteogenic (bone forming in its own right), osteo-inductive and/or osteo-conductive properties.

The term “osteo-inductive” refers to the capacity of a component such as a peptide growth factor to recruit immature cells such as stem cells, MSC and stimulate those cells to differentiate into pre-osteoblasts and mature osteoblasts, thereby forming bone tissue. The pharmaceutical compositions may further comprise a component with osteo-inductive properties such as an osteo-inductive protein or peptide, for instance a bone morphogenetic protein, such as BMP-2, BMP-7 or BMP-4; a hydrogel or biopolymer such as collagen, hyaluronic acid or derivatives thereof, osteonectin, fibrinogen, or osteocalcin. Preferably, the pharmaceutical compositions may further comprise hyaluronic acid or derivatives thereof, collagen or fibrinogen.

The term “osteo-conductive” refers to the ability of a component to serve as a scaffold on which bone cells can attach, migrate, grow and produce new bone. The pharmaceutical compositions may further comprise a component with osteo-conductive properties, for example, an osteo-conductive scaffold or matrix or surface such as without limitation tricalcium phosphate, hydroxyapatite, combination of hydroxyapatite/tricalcium phosphate particles (HA/TCP), gelatine, poly-lactic acid, poly-lactic glycolic acid, hyaluronic acid, chitosan, poly-L-lysine, or collagen.

The pharmaceutical compositions according to the present invention may as mentioned above comprise components useful in the repair of bone wounds and defects. The pharmaceutical compositions may comprise a scaffold or matrix with osteo-conductive properties. The osteoprogenitors or osteoblastic cells or the cell population comprising osteoprogenitors and/or osteoblastic cells may be combined with demineralized bone matrix (DBM) or other matrices to make the composite osteogenic as well as osteo-conductive and osteo-inductive. Similar methods using autologous bone marrow cells with allogeneic DBM have yielded good results (Connolly et al. 1995. Clin Orthop 313: 8-18).

The pharmaceutical compositions according to the present invention can further include or be co-administered with a complementary bioactive factor or osteo-inductive protein such as a bone morphogenetic protein, such as BMP-2, BMP-7 or BMP-4, or any other growth factor. Other potential accompanying components include inorganic sources of calcium or phosphate suitable for assisting bone regeneration (WO 00/07639). If desired, cell preparation can be administered on a carrier matrix or material to provide improved tissue regeneration. For example, the material can be a hydrogel, or a biopolymer such as gelatine, collagen, hyaluronic acid or derivatives thereof, osteonectin, fibrinogen, or osteocalcin. Biomaterials can be synthesized according to standard techniques (e.g., Mikos et al., Biomaterials 14:323, 1993; Mikos et al., Polymer 35:1068, 1994; Cook et al., J. Biomed. Mater. Res. 35:513, 1997).

Without limitation, depending on the type and severity of the disease, a typical daily dosage of GDF-8 might range from about 1 ng/kg to 100 mg/kg of body weight or more. For repeated administrations over several days or longer, depending on the condition, the treatment is sustained until a desired suppression of disease symptoms occurs. A preferred dosage of GDF-8 may be in the range from about 10 ng/kg to about 10 mg/kg of body weight. Thus, one or more doses of about 10 ng/kg, 100 ng/kg, 500 ng/kg, 1 mg/kg or 10 mg/kg (or any combination thereof) may be administered to the patient. Such doses may be administered intermittently, e.g., every week or every two or three weeks.

Without limitation, a typical dose of for instance the cell composition to be administered may range from about 0.05×10⁶ cells to 5×10⁹ cells per injection. For example, the dose to be administered may range from about 0.5×10⁶ cells to 1×10⁹ cells per injection. Preferably, the dose to be administered ranges from about 4×10⁶ cells to 250×10⁶ cells per injection.

As used herein the term “implant” broadly refers to medical devices manufactured to substitute a missing biological structure, support or complement a damaged biological structure, or enhance an existing biological structure. Implants as intended herein may comprise biological component(s), such as cells and/or tissues. Exemplary medical implants include, e.g., pins, rods, screws, plates and other structures used in bone surgery and bone healing.

The term “transplant” broadly refers to transplanted biomedical tissue, e.g., including organ, tissue, or cells.

Alternatively or in addition, the MSC cells or the present osteoprogenitors or osteoblastic cells or the cell population comprising osteoprogenitors and/or osteoblastic cells, or other cell types envisaged herein, may be stably or transiently transformed with a nucleic acid of interest prior to administration to the subject. Nucleic acid sequences of interest include, but are not limited to those encoding gene products that further enhance the growth, differentiation and/or mineralization of osteoblastic cell populations. For example, an expression system for BMP-2, can be introduced into the MSC in a stable or transient fashion for the purpose of treating non-healing fractures or osteoporosis. Methods of transformation of MSC and of osteoprogenitors or osteoblastic cells or a cell population comprising osteoprogenitors and/or osteoblastic cells are known to those skilled in the art.

Also provided are methods of producing said pharmaceutical compositions by admixing the cells or other pharmaceutically active ingredients of the invention with one or more additional components as described above as well as with one or more pharmaceutical excipients as described above.

Also disclosed is an arrangement or kit of parts comprising a surgical instrument or device for administration of the osteoprogenitors or osteoblastic cells or the cell population comprising osteoprogenitors and/or osteoblastic cells, or other cell types, as taught herein or the pharmaceutical compositions as defined herein to a subject, such as for example systemically or locally, for example at a site of musculoskeletal lesion, for example, by injection, and further comprising the osteoprogenitors or osteoblastic cells or the cell population comprising osteoprogenitors and/or osteoblastic cells, or other cell types, as taught herein or the pharmaceutical compositions as defined herein.

For example but without limitation, such arrangement or kit of parts may comprise: a vial with osteoprogenitors or osteoblastic cells or a cell population comprising osteoprogenitors and/or osteoblastic cells, or other cell types, obtainable with the present methods or a vial comprising the osteoprogenitors or osteoblastic cells or the cell population comprising osteoprogenitors and/or osteoblastic cells, or other cell types, as intended herein; and a device for delivering said osteoprogenitors or osteoblastic cells or the cell population comprising osteoprogenitors and/or osteoblastic cells, or other cell types, to a subject and having reservoir means for storing said osteoprogenitors or osteoblastic cells or the cell population comprising osteoprogenitors and/or osteoblastic cells, or other cell types, piston means movable along the longitudinal axis of the reservoir for dispensing said osteoprogenitors or osteoblastic cells or the cell population comprising osteoprogenitors and/or osteoblastic cells, or other cell types, and a hollow needle mounted on said reservoir means for delivering said osteoprogenitors or osteoblastic cells or the cell population comprising osteoprogenitors and/or osteoblastic cells, or other cell types, to the subject.

The osteoprogenitors or osteoblastic cells or the cell population comprising osteoprogenitors and/or osteoblastic cells, or other cell types, can be administered in a manner that permits them to graft or migrate to the intended tissue site and reconstitute or regenerate the functionally deficient area. Administration of the composition will depend on the musculoskeletal site being repaired. For example, osteogenesis can be facilitated in concordance with a surgical procedure to remodel tissue or insert a split, or a prosthetic device such as a hip replacement. In other circumstances, invasive surgery will not be required, and the composition can be administered by injection or (e.g., for repair of the vertebral column) using a guidable endoscope.

In an embodiment the pharmaceutical cell preparation as define above may be administered in a form of liquid or viscous composition. In embodiments, the cells or pharmaceutical composition comprising such can be administered systemically, topically or at a site of lesion.

In another embodiment, the cells or cell populations may be transferred to and/or cultured on suitable substrate to provide for implants. The substrate on which the cells can be applied and cultured can be a metal, such as titanium, cobalt/chromium alloy or stainless steel, a bioactive surface such as a calcium phosphate, polymer surfaces such as polyethylene, and the like. Although less preferred, siliceous material such as glass ceramics, can also be used as a substrate. Most preferred are metals, such as titanium, and calcium phosphates, even though calcium phosphate is not an indispensable component of the substrate. The substrate may be porous or non-porous.

For example, cells that have proliferated, or that are being differentiated in culture dishes, can be transferred onto three-dimensional solid supports in order to cause them to multiply and/or continue the differentiation process by incubating the solid support in a liquid nutrient medium of the invention, if necessary. Cells can be transferred onto a three-dimensional solid support, e.g. by impregnating said support with a liquid suspension containing said cells. The impregnated supports obtained in this way can be implanted in a human subject. Such impregnated supports can also be re-cultured by immersing them in a liquid culture medium, prior to being finally implanted.

The three-dimensional solid support needs to be biocompatible so as to enable it to be implanted in a human. It can be of any suitable shape such as a cylinder, a sphere, a plate, or a part of arbitrary shape. Of the materials suitable for the biocompatible three-dimensional solid support, particular mention can be made of calcium carbonate, and in particular aragonite, specifically in the form of coral skeleton, porous ceramics based on alumina, on zirconia, on tricalcium phosphate, and/or hydroxyapatite, imitation coral skeleton obtained by hydrothermal exchange enabling calcium carbonate to be transformed into hydroxyapatite, or else apatite-wollastonite glass ceramics, bioactive glass ceramics such as Bioglass™ glasses.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as follows in the spirit and broad scope of the appended claims.

The above aspects and embodiments are further supported by the following non-limiting examples.

### EXAMPLES

### Example 1

### Culturing Cells from Subjects

Bone marrow was harvested from iliac crest of human healthy donors. Heparinized bone marrow was subjected to fractionation on a density gradient solution (Ficoll™ plate Premium, GE Healthcare). The density gradient solution allowed purification of mononuclear cells (MNC). MNC were then plated at a density between 5×10³ and 5×10⁵ cells/cm². Cells were cultured in culture medium in the presence of serum and FGF-2. Primary cultures were maintained for 10 to 18 days in culture, with a complete medium change between day 1 and day 5, and changed regularly afterwards. During the primary culture, the non-adherent hematopoietic cells were withdrawn through the medium change, and mesenchymal derived-cells acquired a fibroblast-like morphology. After primary culture, mesenchymal derived-cells were harvested by trypsin treatment, and replated for secondary cell culture. The secondary culture was performed for a period of from 3 days to 18 days.

### Example 2

### Culturing Cells in Medium Comprising Serum, GDF-8 and FGF-2 from Primary Cell Culture

After Ficoll, MNC cells were cultured during primary culture as described in Example 1, but in the presence of GDF-8 (Recombinant human myostatin, Peprotech) or absence of GDF-8 (control). When confluent, cells of primary culture were passaged, replated for secondary culture at different densities and cultured in the presence of GDF-8 (GDF-8) or absence of GDF-8 (control). The concentration of GDF-8 used was between 1 and 100 ng/ml.

At the end of the culture, cells were characterized by their proliferation, phenotype (FACS), protein secretion (IL6, VEGF, Decorin, Osteoprotegerin), mineralization ability and enzymatic ALP activity.

### Effect on Culture Yield

To assess the impact on cell growth of culturing the cells from primary culture in a medium comprising serum, FGF-2 and GDF-8, global culture yield of primary and secondary culture was determined for cells cultured in the presence of GDF-8 (GDF-8) or in the absence of GDF-8 (control).

Culture yield was determined as follows: number of cells harvested at the end of culture or growth factor treatment divided by number of cells plated for culture. Cell viability was assessed by Trypan Blue exclusion method and cell number was determined by counting cell suspension in Bürker chamber.

Primary and secondary culture yield represent cell proliferation during primary and secondary cell cultures, respectively.

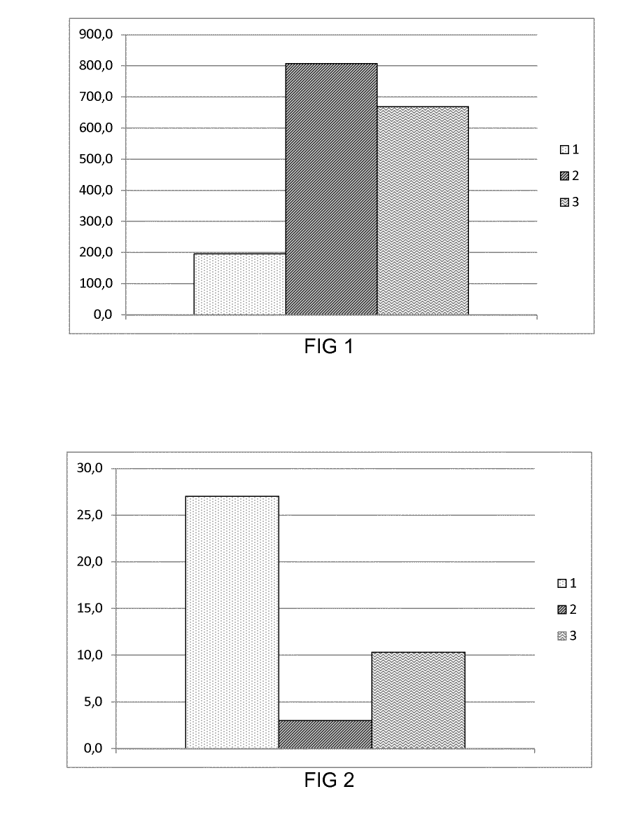

Mean data are presented in Table 1 and FIG. 1. The culture yields showed that culturing cells in a medium comprising serum, FGF-2 and GDF-8 increased cell proliferation compared with culturing cells in such medium without GDF-8 (Table 1, FIG. 1).

TABLE 1
Mean cell culture yields (%) after culturing cells in
the presence of GDF-8 (GDF-8), absence of GDF-8 (control)
or in the presence of TGF-beta 1 (TGF-beta 1)

| | Parameter | Control | GDF-8 | TGF-beta 1 |
|---|---|---|---|---|
| Global culture | Mean ± SD | 196 ± 71 | 669 | 807 ± 621 |
| yield | N | 3 | 1 | 10 |

### Expression of HLA-DR and ALP by FACS

In order to characterize cells, cell phenotype was determined using FACS analysis after detachment of the cells. The measured markers were CD45 (hematopoietic marker), CD105 (mesenchymal marker), ALP (osteogenic marker) and HLA-DR (immunogenic marker).

The cell phenotype was determined by FACS analysis using antibodies against CD45, CD105, ALP and HLA-DR, coupled with fluorescein isothiocyanate (FITC), phycoerythrin (PE), or allophycocyanin (APC) from BD Biosciences (anti-CD45, anti-CD105, anti-HLA-DR) and R&D Systems (anti-ALP). Cells were analyzed with BD FACS Canto II flow cytometer (Becton Dickinson). The expression of the markers is given as a percentage, i.e., as the number of cells expressing the marker divided by the total number of cells scored by FACS.

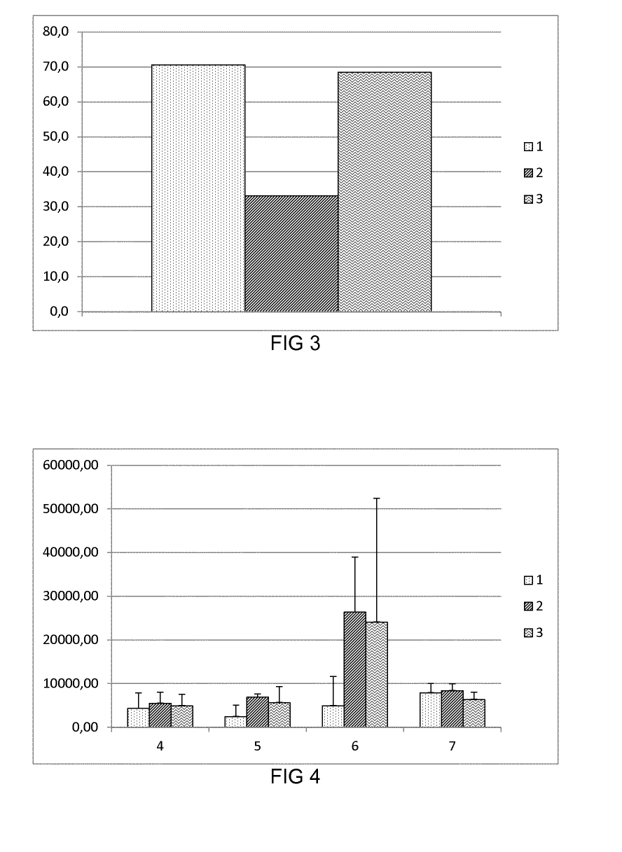

Mean data are presented in Table 2 and FIGS. 2 and 3. The data showed that culturing cells in the presence of GDF-8 decreased HLA-DR expression compared with control (Table 2 and FIG. 2). The results further showed that culturing cells in the presence of GDF-8 maintained a high level of ALP expression in opposition with culturing cells in the presence of TGF-beta 1 (Table 2 and FIG. 3).

The results also demonstrated that culturing cells in the presence of GDF-8 maintained a similar expression of the hematopoietic marker CD45 (Table 2), mesenchymal marker CD105 (Table 2), and other immunogenicity markers CD28/CD80/CD83/CD86 (results not shown) compared with control cells cultured in the absence of GDF-8.

TABLE 2
Expression of markers (%) after culturing cells in the presence of GDF-8 (GDF-
8), absence of GDF-8 (control) or in the presence of TGF-beta 1 (TGF-beta 1)

| Treatment | Parameter | CD45 | CD105 | ALP | HLA-DR |
|---|---|---|---|---|---|
| Control | Mean ± SD | 1.8 ± 0.8 | 98.8 ± 1.7 | 70.5 ± 22.1 | 27.0 ± 7.2 |
| | N | 3 | 3 | 3 | 3 |
| GDF-8 | Mean ± SD | 2.6 ± 0.6 | 99.0 ± 1.3 | 68.4 ± 29.5 | 10.3 ± 3.2 |
| | N | 2 | 2 | 2 | 2 |
| TGF-beta 1 | Mean ± SD | 1.3 ± 0.9 | 86.7 ± 31.1 | 33.1 ± 28.9 | 3.0 ± 4.1 |
| | N | 10 | 10 | 10 | 10 |

### Secretion of Markers in Supernatant

Protein secretion was studied to assess the osteoblastic commitment of cells and their ability to recruit cells involved in bone repair. Protein secretion was studied by ELISA. More particularly, culture supernatants were harvested after culture to assess the production of IL6 and VEGF (cell recruitment), and Decorin and Osteoprotegerin (bone matrix proteins and bone factors).

Secretion of IL6, VEGF, Decorin and Osteoprotegerin was assessed in culture supernatants collected at each medium change. The assays were performed following manufacturer instructions (R&D Systems Human IL6 ELISA kit #DY206, R&D Systems Human VEGF ELISA kit #DY293b, R&D Systems Human Decorin ELISA kit #DY143, R&D Systems Human Osteoprotegerin ELISA kit #DY805). Absorbance was measured with a Multiskan plate reader at 450 nm.

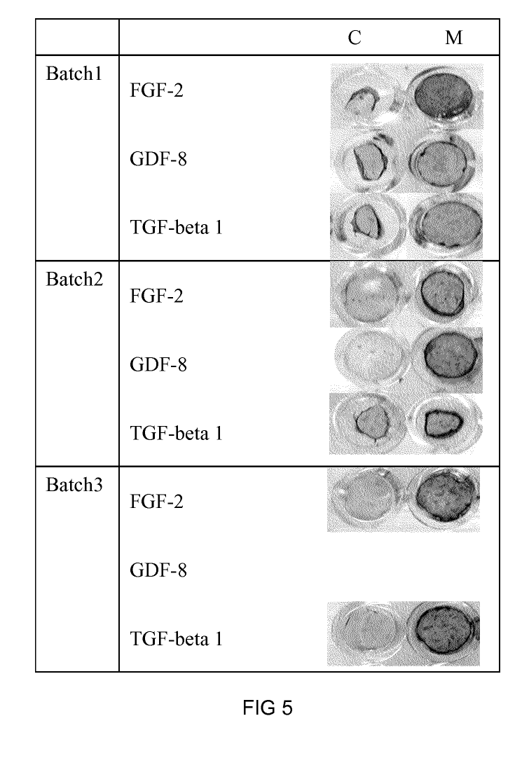

Mean data are presented in Table 3 and in FIG. 4. Levels of IL6, VEGF, Decorin and Osteoprotegerin (OPG) in GDF-8 cultures were similar to those of control or those of TGF-beta 1 cultures (Table 3 and FIG. 4).

TABLE 3
Proteins secretion in supernatant after culturing cells in the presence of GDF-8
(GDF-8), absence of GDF-8 (control) or in the presence of TGF-beta 1 (TGF-beta 1)

| | | IL6 | VEGF | Decorin | OPG |
|---|---|---|---|---|---|
| Treatment | Parameter | (pg/ml) | (pg/ml) | (pg/ml) | (pg/ml) |
| Control | Mean ± SD | 4378 ± 3498 | 2523 ± 2557 | 5008 ± 6613 | 7956 ± 2048 |
| | N | 7 | 7 | 7 | 7 |
| GDF-8 | Mean ± SD | 4969 ± 2585 | 5689 ± 3610 | 24100 ± 28374 | 6412 ± 1630 |
| | N | 2 | 2 | 2 | 2 |
| TGF-beta 1 | Mean ± SD | 5568 ± 2481 | 6942 ± 677 | 26413 ± 12557 | 8449 ± 1522 |
| | N | 3 | 3 | 3 | 3 |

### Mineralization of Secondary and Tertiary Cultures

Mineralization was used to assess the ability of cells to form a mineralized matrix. To this end, cells were plated at the end of secondary culture in 24-well plate at a density between of 5×10³ and 5×10⁴ cells/cm² and cultured in medium comprising serum, FGF-2 and GDF-8 (FGF-2/GDF-8) or in such medium without GDF-8 (FGF-2) or in a medium comprising serum, FGF-2 and TGF-beta 1 (FGF-2/TGF-beta 1). The next day, mineralization was induced by replacing culture medium with osteogenic medium, i.e. alpha-MEM supplemented with 15% FBS, 1% penicillin/streptomycin, beta-glycerophosphate (10 mM), ascorbic acid (vitamin C) (50 μg/ml), and dexamethasone (10⁻⁸ M). Control medium consisted of alpha-MEM supplemented with 15% FBS and 1% penicillin/streptomycin. After 3 weeks of culture, cells were fixed in 4% paraformaldehyde/PBS and stained by Alizarin Red S that stains inorganic calcium deposits. Finally, the mineralization capacity was assessed by a semi-quantitative score ranging from zero for any observed mineralization to 2.5 for the maximal observed mineralization.

Mineralization was observed in all culture conditions including serum, FGF-2 and GDF-8 (FGF-2/GDF-8) or FGF-2 and TGFβ-1 (FGF-2/TGF-beta 1) compared with conditions without GDF-8 (FGF-2) (Table 4 and FIG. 5). Consequently, GDF-8 did not seem to detract from the mineralization capacity of cells cultured with a method illustrating the present invention.

TABLE 4
Semi-quantitative scores of mineralization after culturing
cells in the presence of GDF-8 (GDF-8), absence of GDF-8
(control) or in the presence of TGF-beta 1 (TGF-beta 1)

| | | Control medium | Osteogenic medium |
|---|---|---|---|
| Mean | Control | 0 | 1.25 |
| N = 3 | GDF-8 | 0 | 1.25 |
| | TGF-beta 1 | 0 | 1.2 |

### Alkaline Phosphatase Activity

The activity of alkaline phosphatase, a key enzyme in bone matrix formation, was determined by a biochemical assay based on the hydrolysis of the phosphate group of a synthetic substrate p-Nitrophenyl phosphate (pNPP) and detection of the reaction product at 415 nm. The ALP enzymatic activity of the cells was determined by comparison with a standard curve based on purified calf intestinal alkaline phosphatase activity. The ALP activity was reported in Units of ALP/mg of protein. Protein content was determined by Bradford assay. One unit of ALP hydrolyses 1 μmol of pNPP per min at 37° C.

Cells cultured in the presence of GDF-8 seemed to have a higher ALP activity (Table 5). These results corroborate FACS analysis (Table 2).

TABLE 5
Enzymatic activity of alkaline phosphatase (ALP) after culturing
cells in the presence of GDF-8 (GDF-8), absence of GDF-8 (control)
or in the presence of TGF-beta 1 (TGF-beta 1)

| Treatment | mU/mg stock solution |
|---|---|
| control | 323.49 |
| GDF-8 | 438.13 |
| TGF-beta 1 | 203.69 |

The following non-limiting observations may be made:

Culturing cells from primary culture in a medium comprising serum, FGF-2 and GDF-8 stimulated cell proliferation compared with culturing cells in such medium without GDF-8.

Culturing cells from primary culture in a medium comprising serum, FGF-2 and GDF-8 decreased the expression of HLA-DR. Consequently, GDF-8 reduced the immunogenicity of cells.

Cells cultured in a medium comprising serum, FGF-2 and GDF-8 displayed a similar osteogenic phenotype as control cells cultured in such medium without GDF-8. Culturing cells in a medium comprising serum, FGF-2 and GDF-8 did not seem to affect ALP expression during culture. Furthermore, culturing cells in a medium comprising serum, FGF-2 and GDF-8 did not affect the secretion of bone matrix proteins such as decorin and osteoprotegerin and proteins involved in cells recruitment such as VEGF. In addition, cells cultured in a medium comprising serum, FGF-2 and GDF-8 were able to synthesize a mineralized matrix.

### Example 3

### Culturing Cells in Medium Comprising Serum, GDF-8 and FGF-2 from Tertiary Culture

To explore the effect of culturing cells in a medium comprising serum, FGF-2 and GDF-8 on cells cultured during primary and secondary cultures with FGF-2 as set forth in Example 1, the cells were plated in 6-well plate for tertiary culture and left for 24 hours to allow attachment to the substrate surface. The next day, cells were cultured in medium comprising serum and FGF-2 and in the presence of GDF-8 (GDF-8) or in the absence of GDF-8 (control).

Cell Phenotype after 4 Days of Culture

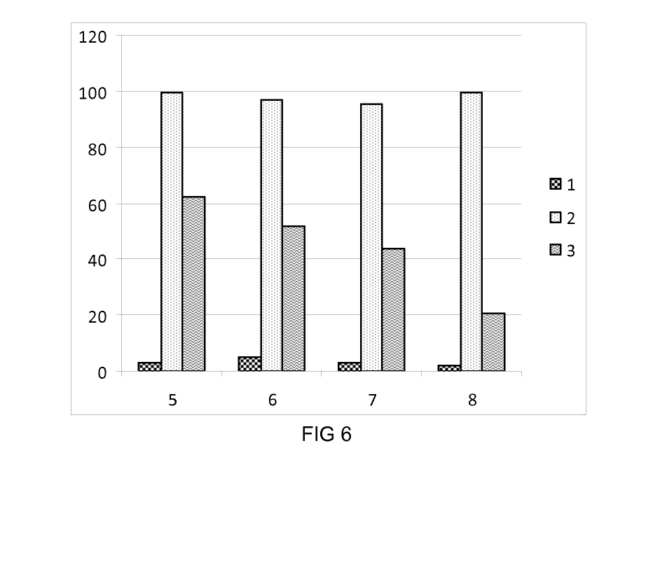

The phenotype of cells cultured from tertiary culture for 4 days in medium comprising serum, FGF-2 and GDF-8, was measured by FACS as described herein. The expression of the markers is given as a percentage of positive cells, i.e. as the number of cells expressing the marker divided by the total number of cells plated for culture. The expression of the hematopoietic marker CD45 and the mesenchymal marker CD105 were similar in all conditions (Table 6 and FIG. 6). As shown in Table 6 and FIG. 6, the expression of HLA-DR was lower after culturing cells from tertiary culture for 4 days in medium comprising serum, FGF-2 and GDF-8 compared with culturing cells from tertiary culture for 4 days in medium comprising serum, FGF-2 and TGF-beta 1. In conclusion, using a method illustrating the present invention may advantageously improve the characteristics of the obtained cells or cell populations.

TABLE 6
Expression of markers (%) before and after culturing cells
for 4 days in the presence of GDF-8 (GDF-8), absence of GDF-8
(control) or in the presence of TGF-beta 1 (TGF-beta 1)

| Treatment | CD45 | CD105 | HLA-DR |
|---|---|---|---|
| Control before culturing | 3 | 99.3 | 62.2 |
| Control [symbol] 4 days [symbol] | 5.2 | 96.8 | 51.7 |
| GDF-8 100 ng/ml [symbol] 4 days [symbol] | 2.2 | 99.6 | 20.8 |
| TGF-beta 1 [symbol] 4 days [symbol] | 3.1 | 95.3 | 43.5 |

Cell Phenotype after 6 Days of Culturing with Different Concentrations of GDF-8

Cells were also cultured from tertiary culture for 6 days in medium comprising serum, FGF-2 and GDF-8, wherein GDF-8 was present in the medium in increasing doses of 50 ng/ml, 100 ng/ml or 200 ng/ml.

The phenotype of cells cultured from tertiary culture for 6 days in medium comprising serum, FGF-2 and GDF-8, was analyzed by FACS as described herein. The expression of the markers is given as a percentage of positive cells, i.e. as the number of cells expressing the marker divided by the total number of cells plated for culture.

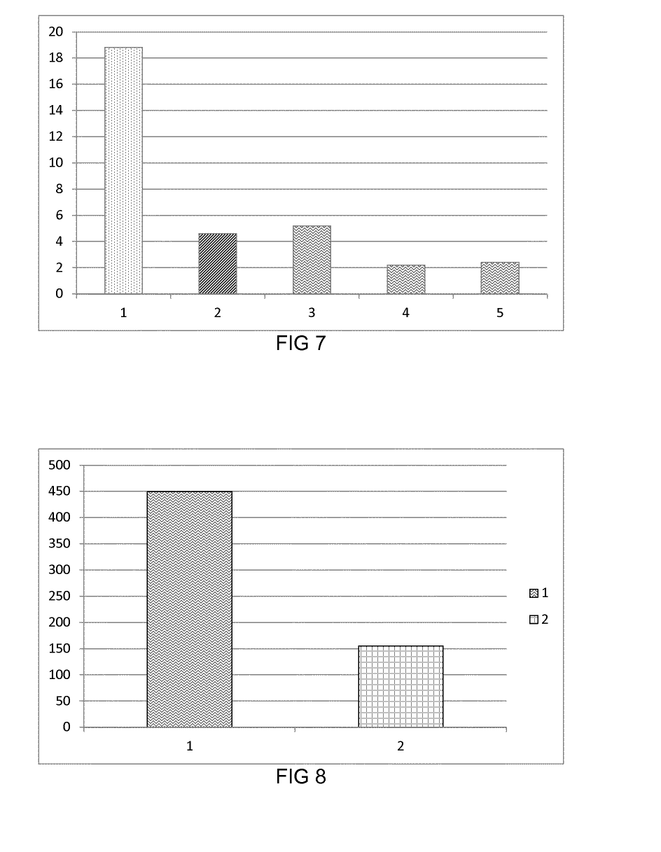

In all cell culture conditions from tertiary culture for 6 days in medium comprising serum, FGF-2 and GDF-8, HLA-DR expression was lower than in control condition. Furthermore, HLA-DR expression seemed to decrease with increasing concentrations of GDF-8 (Table 7 and FIG. 7).

TABLE 7
Expression of markers (%) after culturing cells for 6 days
in the presence of GDF-8 (GDF-8), absence of GDF-8 (control)
or in the presence of TGF-beta 1 (TGF-beta 1)

| Treatment | HLA-DR |
|---|---|
| Control | 18.8 |
| GDF-8 50 ng/ml | 5.2 |
| GDF-8 100 ng/ml | 2.2 |
| GDF-8 200 ng/ml | 2.4 |
| TGF-beta 1 1 ng/ml | 4.6 |

The following non-limiting observations can be made:

Culturing cells from tertiary culture in a medium comprising serum, FGF-2 and GDF-8, induced a decrease in HLA-DR expression of the cells. The decrease in HLA-DR expression was more pronounced with higher concentrations of GDF-8.

### Example 4

### Culturing Cells in Medium Comprising Serum and GDF-8 from Primary Culture

Bone marrow cells were cultured in a medium comprising (1) serum, FGF-2 and GDF-8 or (2) serum and GDF-8. After primary culture, cells were passaged and replated for secondary culture in corresponding media. The concentration of GDF-8 used was 100 ng/ml. At the end of the culture, cells were characterized by their proliferation and phenotype (FACS).

### Effect on Culture Yield

Culture yields were determined by cell count. Data are presented in Table 8 and FIG. 8. Data showed that culture yields were decreased in the absence of FGF-2 in the medium compared to culture in presence of FGF-2 with or without GDF-8.

TABLE 8
Global culture yields (%) after culturing cells from primary
cultures in medium containing GDF-8 with or without FGF-2

| Treatment | Global culture yield |
|---|---|
| GDF-8 100 ng/ml + FGF-2 | 450 |
| GDF-8 100 ng/ml | 155 |

In additional experiments of the same kind, the number of samples has been increased, producing the results captured in Table 9.

TABLE 9
Mean Global culture yields (%) after culturing cells from primary
cultures in medium containing GDF-8 with or without FGF-2

| Treatment | Global culture yield |
|---|---|
| GDF-8 100 ng/ml + FGF-2 | 730 ± 736 (n = 7) |
| GDF-8 100 ng/ml | 142 ± 99 (n = 4) |

### Expression of HLA-DR and ALP by FACS

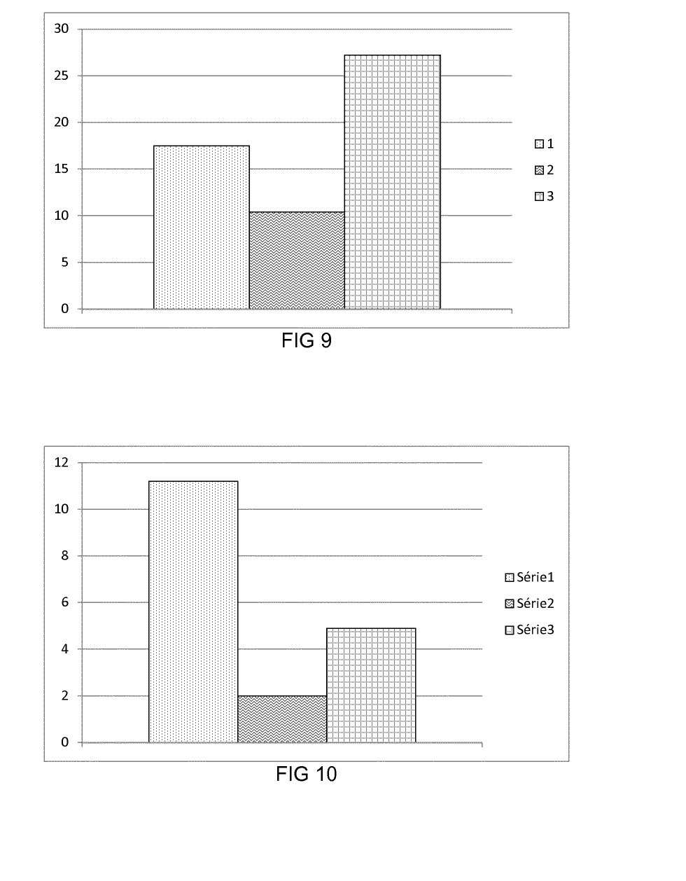

The cell phenotype after primary and secondary culture was determined by FACS analysis as described in Example 2. The expression of the markers ALP and HLA-DR is given in percentage of positive cells. ALP expression levels in primary cultures comprising GDF-8 were similar in cultures in the presence or absence of FGF-2, whereas in secondary cultures, a decrease in ALP expression was observed in cultures comprising FGF-2 compared with cultures without FGF-2 (Table 10 and FIG. 9). Cultures in media comprising GDF-8 showed a decrease in HLA-DR expression, with or without FGF-2, in comparison with cultures in medium comprising FGF-2 without GDF-8. The decrease was more pronounced in the presence of GDF-8 and FGF-2 (Table 10 and FIG. 10).

TABLE 10
Expression of markers (%) after culturing cells
in medium comprising FGF-2 (control) or in medium
comprising GDF-8 with or without FGF-2

| Treatment | ALP | HLA-DR |
|---|---|---|
| Control | 17.5 | 11.2 |
| GDF-8 + FGF-2 | 10.4 | 2 |
| GDF-8 | 27.2 | 4.9 |

In additional experiments of the same kind, the number of samples has been increased, producing the results captured in Table 11.

TABLE 11
Expression of markers (%) after culturing cells
in medium comprising FGF-2 (control) or in medium
comprising GDF-8 with or without FGF-2

| Treatment | ALP | HLA-DR |
|---|---|---|
| Control | 32 ± 16 (n = 9) | 9 ± 11 (n = 9) |
| GDF-8 + FGF-2 | 26 ± 11 (n = 7) | 2 ± 1 (n = 7) |
| GDF-8 | 34 ± 6 (n = 3) | 3 ± 2 (n = 3) |

The following non-limiting observations can be made:

As expected, absence of the growth factor FGF-2 in culture medium of cells decreased the primary and secondary culture yield, but did not affect decrease in HLA-DR expression of cells. Presence of GDF-8 in culture medium of cells was able to induce osteoblastic differentiation of MSCs with a low immunogenicity profile, independently of the presence of FGF-2.

Collectively, the above examples show that culturing cells in a medium comprising serum, FGF-2 and GDF-8 increased cell growth, decreased HLA-DR expression and did not reduce osteogenic differentiation compared with culturing cells from primary culture in such medium without GDF-8. In particular, culturing cells from primary culture in a medium comprising serum, FGF-2 and GDF-8 did not alter ALP expression and the expression of bone matrix proteins such as decorin and osteoprotegerin or of proteins involved in cell recruitment such as VEGF.

In addition, the above examples show that culturing cells from tertiary culture in a medium comprising serum, FGF-2 and GDF-8 had no effect on ALP expression compared with culturing cells from primary culture in such medium without GDF-8. Advantageously, it has been observed that culturing cells from secondary or tertiary culture in a medium comprising serum, FGF-2 and GDF-8 decreased HLA-DR expression, in particular at longer treatments compared with culturing cells from primary culture in such medium without GDF-8.

### Example 5

### Culturing Cells in Medium Comprising GDF-8

Bone marrow is harvested from iliac crest of human healthy donors. Bone marrow is subjected to fractionation on a density gradient solution (Ficoll™ plate Premium, GE Healthcare). The density gradient solution allows purification of mononuclear cells (MNC). MNC are then plated at a density between 5×10³ and 5×10⁵ cells/cm² and grown according to a standard MSC culture protocol. The following experiments are performed:

The MSC are subjected to a standard chondrocytic lineage differentiation protocol, either with or without GDF-8 added in the culture medium from primary or secondary culture or, where applicable, tertiary culture. The resulting cells of chondrocytic lineage, in particular chondroblasts and chondrocytes, display decreased HLA-DR expression when GDF-8 is included compared to when GDF-8 is not included.

The MSC are subjected to a standard adipocytic lineage differentiation protocol, either with or without GDF-8 added in the culture medium from primary or secondary culture or, where applicable, tertiary culture. The resulting cells of adipocytic lineage, in particular adipoblasts and adipocytes, display decreased HLA-DR expression when GDF-8 is included compared to when GDF-8 is not included.

The MSC are subjected to a standard myocytic lineage differentiation protocol, either with or without GDF-8 added in the culture medium from primary or secondary culture or, where applicable, tertiary culture. The resulting cells of myocytic lineage, in particular myoblasts and myocytes, display decreased HLA-DR expression when GDF-8 is included compared to when GDF-8 is not included.

The MSC are subjected to a standard tendonocytic lineage differentiation protocol, either with or without GDF-8 added in the culture medium from primary or secondary culture or, where applicable, tertiary culture. The resulting cells of tendonocytic lineage, in particular tenoblasts and tenocytes, display decreased HLA-DR expression when GDF-8 is included compared to when GDF-8 is not included.

The MSC are subjected to a standard stromogenic lineage differentiation protocol, either with or without GDF-8 added in the culture medium from primary or secondary culture or, where applicable, tertiary culture. The resulting cells of stromogenic lineage, in particular stromal cells, display decreased HLA-DR expression when GDF-8 is included compared to when GDF-8 is not included.

1. A method for reducing the immunogenicity of cells in vitro, the method comprising sing cells to growth and differentiation factor 8 (GDF-8), wherein the cells are selected from the group consisting of mesenchymal stem cells (MSC), cells obtained by differentiation of MSC, cells of osteocytic lineage, cells of chondrocytic lineage, cells of adipocytic lineage, cells of myocytic lineage, cells of tendonocytic lineage, cells of fibroblastic lineage, and cells of stromogenic lineage.

2. The method according to claim 1, wherein the cells of osteocytic lineage, cells of chondrocytic lineage, cells of adipocytic lineage, cells of myocytic lineage, cells of tendonocytic lineage, cells of fibroblastic lineage, or cells of stromogenic lineage are obtained by differentiation of MSC.

3. The method according to claim 1, wherein the cells are MSC, osteoprogenitors, osteoblastic cells, osteocytes, chondroblastic cells, chondrocytes, adipoblastic cells, adipocytes, myoblastic cells, or myocytes, preferably wherein the cells are MSC, osteoprogenitors, osteoblastic cells, chondroblastic cells, or chondrocytes.

4. The method according to claim 1, wherein the cells are MSC.

5. The method according to claim 1, wherein the cells are osteoprogenitors or osteoblastic cells.

6. The method according to claim 1, wherein the cells are mammalian cells such as human cells or non-human mammalian cells, preferably human cells.

7. The method according to claim 1, wherein GDF-8 reduces MHC class II cell surface receptor complex on the cells and optionally reduces one or more costimulatory factors on the cells.

8. The method according to claim 7, wherein on human cells the MHC class II cell surface receptor is human leukocyte antigen DR (HLA-DR).

9. The method according to claim 1, wherein the cells are comprised in an implant or transplant, preferably in an osseous and/or articular tissue implant or transplant, or in a pharmaceutical formulation.

10. A method for reducing the immunogenicity of cells with GDF-8 in vivo in a subject, the method comprising exposing cells in a subject to GDF-8 in vivo, wherein the cells are selected from the group consisting of MSC, cells obtained by differentiation of MSC, cells of osteocytic lineage, cells of chondrocytic lineage, cells of adipocytic lineage, cells of myocytic lineage, cells of tendonocytic lineage, cells of fibroblastic lineage, and cells of stromogenic lineage.

11. The method according to claim 10, wherein GDF-8 and the cells are administered to the subject in combination.

12. The method according to claim 10, wherein the cells comprise MSC, osteoprogenitors, osteoblastic cells, osteocytes, chondroblastic cells, chondrocytes, adipoblastic cells, adipocytes, myoblastic cells, or myocytes, preferably wherein the cells are MSC, osteoprogenitors, osteoblastic cells, chondroblastic cells, or chondrocytes.

13. The method according to claim 10, wherein the cells are allogeneic to the subject.

14. A method for treating a musculoskeletal disease in a patient in need thereof, the method comprising administering a combination of GDF-8 and cells to the patient,
wherein the cells are selected from the group consisting of MSC, cells obtained by differentiation of MSC, cells of osteocytic lineage, cells of chondrocytic lineage, cells of adipocytic lineage, cells of myocytic lineage, cells of tendonocytic lineage, cells of fibroblastic lineage, and cells of stromogenic lineage.

15. The method according to claim 14, wherein the cells comprise MSC, osteoprogenitors, osteoblastic cells, osteocytes, chondroblastic cells, chondrocytes, adipoblastic cells, adipocytes, myoblastic cells, or myocytes, preferably wherein the cells are MSC, osteoprogenitors, osteoblastic cells, chondroblastic cells, or chondrocytes.

16. The method according to claim 14, wherein the musculoskeletal disease is a bone disease or an osteoarticular disease, and wherein the cells are selected from the group consisting of MSC, cells of osteocytic lineage, cells of chondrocytic lineage, cells of myocytic lineage, and cells of tendonocytic lineage, preferably wherein the cells are MSC, osteoprogenitors, osteoblastic cells, osteocytes, chondroblastic cells, chondrocytes, myoblastic cells, or myocytes.

17. The method according to claim 14, wherein the cells are allogeneic to the patient to whom they are to be administered.

18. A method of reducing the risk of rejection by a patient of a material administered, implanted, or transplanted into the patient, the method comprising co-administering GDF-8 and the material to the patient.

19. A method according to claim 18, wherein the material comprises osseous and/or articular tissue.

20. A pharmaceutical composition comprising a material to be administered, implanted, or transplanted into a patient and GDF-8, and optionally further comprising one or more pharmaceutically acceptable excipients.

21. The pharmaceutical composition according to claim 20, wherein the material comprises cells selected from the group consisting of MSC, cells obtained by differentiation of MSC, cells of osteocytic lineage, cells of chondrocytic lineage, cells of adipocytic lineage, cells of myocytic lineage, cells of tendonocytic lineage, cells of fibroblastic lineage, and cells of stromogenic lineage.

22. The pharmaceutical composition according to claim 20, wherein the material comprises osseous and/or articular tissue.

23. The method according to claim 14, wherein the musculoskeletal disease is a bone disease or an osteoarticular disease.
